(19) United States
(12) Patent Application Publication
(10) Pub. No.: US 2022/0054607 A1
(43) Pub. Date: Feb. 24, 2022

(54) GLIAL FIBRILLARY ACIDIC PROTEIN TARGETING IMMUNO- AND APTAMER-BASED-THERAPY FOR NEUROINJURY, NEURODEGENERATION, NEURO-DISEASE, AND NEURO-REPAIR

(71) Applicant: UNIVERSITY OF FLORIDA RESEARCH FOUNDATION, INCORPORATED, Gainesville, FL (US)

(72) Inventors: Kevin Ka Wang WANG, Gainesville, FL (US); Zhihui YANG, Gainesville, FL (US); Tian ZHU, Gainesville, FL (US)

(21) Appl. No.: 17/413,835

(22) PCT Filed: Dec. 13, 2019
(86) PCT No.: PCT/US19/66284

§ 371 Date: Jun. 14, 2021

Related U.S. Application Data
(60) Provisional application No. 62/779,163, filed on Dec. 13, 2018.

(51) Int. Cl. A61K 39/00; A61K 38/17; C07K 16/18; A61P 25/28

(52) U.S. Cl. CPC A61K 39/0007; A61K 38/1709; A61K 2039/505; A61P 25/28; C07K 16/18

(57) ABSTRACT

Traumatic brain injury (TBI) is a leading cause of mortality and morbidity around the world. Active immunization with GFAP protein or GFAP peptide or passive immunization with anti-GFAP antibodies or treatment with a GFAP-binding aptamer can be used to reduce the post-TBI induced expression of GFAP, Tau and p-Tau in brain cortex tissues to attenuate the increased serum levels of GFAP after brain injury, and reduce the serum levels of pNF-H, Tau and p-Tau TBI. In addition, GFAP immunization can alleviate anxiety behavior and improve cognitive performance post-injury. Thus, active or passive GFAP immunization or administration of GFAP-binding aptamer(s) provides a treatment with therapeutic value in suppressing astroglial activation/astrogliosis, and in treating neural injuries such as traumatic brain injury, stroke, spinal cord injury, cerebral hemorrhage, or neurodegenerative diseases such as chronic traumatic encephalopathy, Alzheimer's disease, Parkinson's disease, Huntington's disease, multiple sclerosis, amyotrophic lateral sclerosis, frontotemporal dementia, and other dementias.

### STATEMENT OF GOVERNMENT INTEREST

This invention was made with Government support under No. NS085455, awarded by the National Institutes of Health. The Government has certain rights in the invention.

### BACKGROUND OF THE INVENTION

There are several main conditions related to neuroinjury, including traumatic brain injury (TBI), stroke (ischemic and hemorrhagic), spinal cord injury (SCI), and brain hemorrhage (intracerebral hemorrhage, subarachnoid hemorrhage). TBI is a leading cause of mortality and morbidity around the world with a broad spectrum of symptoms and disabilities. There are approximately 1.7-2.0 million incidents of TBI annually. Among all ages, unintentional injuries are the fourth leading cause of death, with over 136,000 lives lost annually. Millions of others suffer a non-fatal injury each year. Neuroinjury also can manifest in the form of neurodegeneration. For example, TBI is also a risk factor for Parkinson's disease, Alzheimer's disease (AD), dementia and multiple sclerosis (MS), and chronic traumatic encephalopathy (CTE).

Also, regardless of the cause or severity of TBI, even mild TBI appears to be a significant risk factor for later development of neurodegenerative diseases such as chronic traumatic encephalopathy (CTE) and other forms of dementia, including AD. In addition, there are other forms of neuroinjury, neurodegeneration or neuro-repair conditions such as spinal cord injury (SCI), frontal temporal dementia (FTD) and other forms of tauopathies or dementia, multiple sclerosis (MS), stroke (ischemic and hemorrhagic), glioblastoma, vanishing white matter disease, and brain hemorrhage (intracerebral hemorrhage, subarachnoid hemorrhage), Parkinson's disease (PD), Alzheimer's disease (AD), Alexander disease, chronic traumatic encephalopathy (CTE), epilepsy, Huntington's disease (HD), amyotrophic lateral sclerosis (ALS), hypoxic ischemic encephalopathy (HIE), neural damage due to drug or alcohol use or abuse (e.g., from amphetamines, ecstasy (3,4-methylenedioxymethamphetamine (MDMA), or ethanol), prion-related disease, peripheral neuropathy, diabetic neuropathy, and chemotherapy-induced neuropathy and neuropathic pain. However, to date, there are still no FDA-approved therapies to treat any forms of TBI. Similarly, there are few treatment options for most of the above described forms of neuroinjury and neurodegenerative conditions.

Astroglia cells are the major and perhaps most abundant cell types in the brain. In healthy brain, astrocytes help with providing structural and network support for neurons and interface with the brain vasculature, including the blood-brain-barrier. Functionally, astrocytes are involved with providing neurotrophic factors (such as glial derived neurotrophic factor (GDNF)), and cytokine/chemokine release that influences the global and local inflammatory response environments, as well as working closely with neurons involved in the glutamate-glutamine synthesis/recycling pathway.

On the other hand, it has been well documented that following neuropertrubation, neuroinjury or when the brain is undergoing neurodegenerative conditions, there is a robust activation of astrocytes (astrogliosis or gliosis). Gliosis can occur in two forms: astrocyte hypertrophy (activation of astroglia with larger, thicker and longer processes) and astrocyte proliferation. Importantly, the astroglia-specific intermediate filament protein called glial fibrillary acidic protein (GFAP) is a critical protein essential for both glial hypertrophy as well as for glial proliferation/cell growth and maturation. Gliosis occurring in a controlled manner might be beneficial following CNS perturbation, but overactivation of the gliosis process is known to have negative impacts on brain recovery or to contribute actively to the neurodegenerative process. In addition to traumatic brain injury, astroglial activation or astrogliosis and GFAP induction might also be involved in the neuro-injury or neuro-repair processes such as SCI, FTD and other forms of tauopathies or dementia, MS, stroke (ischemic and hemorrhagic), glioblastoma, vanishing white matter disease, and brain hemorrhage (intracerebral hemorrhage, subarachnoid hemorrhage), PD, AD, CTE, epilepsy, HD, Alexander disease, ALS, HIE, neural damage due to drug or alcohol use or abuse (e.g., from amphetamines, ecstasy/MDMA, or ethanol), prion-related disease, peripheral neuropathy, diabetic neuropathy, and chemotherapy-induced neuropathy and neuropathic pain.

For example, in injured brain activated astroglia in conjunction with fibroblast overgrowth can form a “glial scar” that prevents neuron synaptic reconnection and hinders functional recovery. Similarly, hyperactivated astrogliosis alone or in conjunction with microglia and infiltrating microphages and T cells can evoke an overactivated and sustained neuroinflammatory response that can cause neuronal or oligodendrocyte injury, death or damage to the extracellular matrix.

Lastly, under neuroinjury or neurodegenerative and neuro-repair conditions, astroglia cells also can be injured or die. Under these conditions, GFAP (50 kDa; a-isoform) is processed by cellular proteases such as calpain and caspase-3, and -6, forming C- and N-terminal truncated forms of GFAP with apparent molecular weights of about 44 kDa, 42 kDa, 40 kDa and 38 kDa. The 38 kDa GFAP breakdown product (GBDP-38K) appears to be the major form truncated form. It has been shown that GFAP and GBDPs include GBDP38K are released into extracellular space, including the extracellular fluid and cerebrospinal fluid (CSF), eventually reaching the circulation. Indeed, full length GFAP protein, as well as GFAP fragments (GBDPs) might be cytotoxic or neurotoxic in cell culture conditions and/or in vivo. Furthermore, GFAP under specific conditions and with posttranslational modifications also can form protein oligomeric aggregates, which can be cytotoxic as well as trigger neurodegeneration. Taken together, these GFAP and GFAP-BDP (GBDP) can be neurotoxic and a contributor of neurodegeneration.

Overall, there is a need in the art for treatments useful for neurological injuries, damage and degeneration in conditions such as TBI, SCI, stroke, CTE, AD, PD and MS and other neurodiseases. Also, in a cell culture model, GFAP-antibody is protective to oxidatively stressed neuroretinal cells. See reference 66, below. GFAP antibody also have neuroprotective effects on retinal ganglion cells in a retina organ culture. See reference 5, below.

### SUMMARY OF THE INVENTION

Therefore, this invention relates to GFAP protein or GBDP direct immunotherapy or aptamer-based therapy for reducing neural injury and neurodegeneration while facilitating neurorecovery. The studies presented in this application investigated an immunotherapeutic approach for neurodegenerative diseases. Brain has been considered exempt from systemic immune surveillance, but there is an ongoing dialogue between the brain and the immune system in which circulating immune cells play a role in brain tissue maintenance and repair. The invention claimed herein relates to a method for treating neuroinjury by active immunization with glial fibrillary acidic protein (GFAP) or passive immunization with anti-GFAP antibodies or treatment with GFAP-binding aptamers.

Specifically, the invention provides a method of suppressing astrogliosis in a subject in need thereof that involves administering glial fibrillary acidic protein (GFAP) or a fragment or breakdown product thereof, or an anti-GFAP antibody or GFAP-binding aptamer.

Another embodiment pertains to a method of treating a brain injury accompanied by astrogliosis in a subject in need thereof, that involves administering glial fibrillary acidic protein (GFAP) or a fragment or breakdown product thereof, or an anti-GFAP antibody or GFAP-binding aptamer.

According to certain embodiments, the subject suffers from traumatic brain injury, stroke, spinal cord injury, cerebral hemorrhage, chronic traumatic encephalopathy, Alzheimer's disease, Parkinson's disease, Huntington's disease, multiple sclerosis, amyotropic lateral sclerosis, frontotemporal dementia, tauopathy diseases, dementias, glioblastoma, vanishing white matter disease, epilepsy, hypoxic ischemic encephalopathy (HIE), neural damage due to drug or alcohol use or abuse, prion-related disease, peripheral neuropathy, diabetic neuropathy, and chemotherapy-induced neuropathy and neuropathic pain.

When the subject suffers from drug abuse, a specific embodiment of drug abuse relates to abuse of amphetamines or ecstasy (MDMA).

Another embodiment pertains to a pharmaceutical composition for immunization of a subject that has or is suspected of having astrogliosis, comprising:

(a) glial fibrillary acidic protein (GFAP) or a fragment thereof, an anti-GFAP antibody or a GFAP-binding aptamer; and

(b) a pharmaceutically acceptable carrier.

In non-limiting embodiments, the brain injury is caused by trauma and/or a neurodegenerative disease. In a specific embodiment, the composition contains GFAP. In another embodiment, the pharmaceutical composition contains an anti-GFAP antibody. Further still, in another embodiment the pharmaceutical composition contains a GFAP-binding aptamer.

According to a further embodiment, disclosed is a method of improving cognitive function in a subject in need thereof that involves administering glial fibrillary acidic protein (GFAP) or a fragment or breakdown product thereof, or an anti-GFAP antibody or GFAP-binding aptamer. Typically, the subject in need will be one that suffers from traumatic brain injury, stroke, spinal cord injury, cerebral hemorrhage, chronic traumatic encephalopathy, Alzheimer's disease, Parkinson's disease, Huntington's disease, multiple sclerosis, amyotropic lateral sclerosis, frontotemporal dementia, tauopathy diseases, dementias, glioblastoma, vanishing white matter disease, epilepsy, hypoxic ischemic encephalopathy (HIE), neural damage due to drug or alcohol use or abuse, prion-related disease, peripheral neuropathy, diabetic neuropathy, and chemotherapy-induced neuropathy and neuropathic pain.

Yet another embodiment pertains to a method of reducing GBDP in a subject in need thereof. The method involves administering glial fibrillary acidic protein (GFAP) or a fragment or breakdown product thereof, or an anti-GFAP antibody or GFAP-binding aptamer. In a specific embodiment, the subject in need suffers from traumatic brain injury.

Also disclosed is a method of attenuating P-Tau/Total ratio in brain tissue associated with a traumatic brain injury in a subject, the method comprising administering glial fibrillary acidic protein (GFAP) or a fragment or breakdown product thereof, or an anti-GFAP antibody or GFAP-binding aptamer.

A further embodiment pertains to a method of reducing circulatory Tau associated with a traumatic brain injury in a subject. The method pertains to administering glial fibrillary acidic protein (GFAP) or a fragment or breakdown product thereof, or an anti-GFAP antibody or GFAP-binding aptamer.

### BRIEF DESCRIPTION OF THE DRAWINGS

The following drawings form part of the present specification and are included to further demonstrate certain embodiments of the present invention. The invention may be better understood by reference to one or more of these drawings in combination with the detailed description of specific embodiments presented herein.

FIG. 1 is a flow chart showing treatment of mice with GFAP protein prior to cortical control impact (CCI) surgery.

FIG. 2A is a set of representative images from manifold immunoblot results. FIG. 2B is a graph showing anti-GFAP titers in mice after immunization.

FIG. 3 shows manifold immunoblotting (FIG. 3A), ELISA (FIG. 3B), and non-immunized mice (FIG. 3C) results as indicated.

FIG. 4 shows GFAP expression in ipsilateral cortex (FIG. 4A), GFAP expression in ipsilateral hippocampus (FIG. 3B) and serum GFAP levels (FIG. 4C) after TBI.

FIG. 5 shows changes in pNF-H levels after brain injury with pre-injury immunization in ipsilateral cortex (FIG. 5A), ipsilateral hippocampus (FIG. 5B), and serum (FIG. 5C).

FIG. 6 shows the effect of pre-injury GFAP immunization on NSE levels in CCI mice in ipsilateral cortex (FIG. 6A), ipsilateral hippocampus (FIG. 6B), and serum (FIG. 6C) as indicated.

FIG. 7 shows the effect of pre-injury immunization with GFAP on tauopathy-linked neurodegeneration. FIG. 7A, FIG. 7C, and FIG. 7E show T-Tau levels, P-Tau levels, and P-Tau/T-Tau ratio, respectively, in ipsilateral cortex as indicated; FIG. 7B, FIG. 7D, and FIG. 7F show T-Tau levels, P-Tau levels, and P-Tau/T-Tau ratio, respectively, in ipsilateral hippocampus as indicated.

FIG. 8A and FIG. 8B show the effect of pre-immunization with GFAP on histopathological outcomes.

FIG. 9A and FIG. 9B show the effect of GFAP immunization on alleviation of post-injury anxiety.

FIG. 10A and FIG. 10B show the effect of GFAP immunization on cognitive functions: memory (FIG. 10A) and spatial learning (FIG. 10B).

FIG. 11 shows that GFAP and the calpain truncated GFAP breakdown product (GBDP-38K) are cytotoxic to primary neurons (using rat cerebrocortical culture), as measured by mitochondria function assay (MTT).

FIG. 12A, FIG. 12B and FIG. 12C show the effects of anti-GFAP MAb therapy on anxiety like behavior. FIG. 12A shows a graph of distance traveled by control and treated mice.

FIG. 12B shows velocity of mouse movement of control and treated mice. FIG. 12C shows time spent in open arms of control and treated mice.

FIG. 13 shows effects of anti-GFAP MAb therapy on cognitive function and memory using a Y-maze setup. FIG. 13A provides a diagram of the Y-maze set up used for the test. FIG. 13B shows time spent in the novel arm and other arms of the Y-maze test for control and treated animals.

FIG. 14 shows effects of anti-GFAP MAb therapy on cognitive function and memory using a Marris Water Maze (MWM) setup. FIG. 14A shows distance moved related to cues training for control and treated mice. FIG. 14B shows distance moved related to spatial learning for control and treated mice. FIG. 14C shows time spent in target quadrant for control and treated mice.

FIG. 15 shows effects of anti-GFAP MAb therapy on GFAP and GBDP levels in Ipsilateral cortex and Ipsilateral hippocampus. FIG. 15A shows western blot indicating GFAP and GDBP levels. FIG. 15B provides a graph providing an indication of GFAP and GDBP levels.

FIG. 16 shows effects of anti-GFAP MAb therapy on p-Tau/Tau ratio at day 30 post antibody immunization.

FIG. 17 shows effects of anti-GFAP MAb therapy on serum Tau levels.

### DETAILED DESCRIPTION

### 1. Definitions

Unless otherwise defined, all technical and scientific terms used herein are intended to have the same meaning as commonly understood in the art to which this invention pertains and at the time of its filing. Although various methods and materials similar or equivalent to those described herein can be used in the practice or testing of the present invention, suitable methods and materials are described below. However, the skilled should understand that the methods and materials used and described are examples and may not be the only ones suitable for use in the invention. Moreover, it should also be understood that as measurements are subject to inherent +variability, any temperature, weight, volume, time interval, pH, salinity, molarity or molality, range, concentration and any other measurements, quantities or numerical expressions given herein are intended to be approximate and not exact or critical figures unless expressly stated to the contrary. Unless defined otherwise, all technical and scientific terms used herein have the same meaning as commonly understood by one of ordinary skill in the art.

As used herein, the term “subject in need thereof” refers to a mammal having a brain injury or suspected of having a brain injury, and includes human patients who have or are suspected of having physical trauma to the brain (e.g., mild, moderate or severe trauma, closed head injury, skull fracture, repeated trauma, and the like) and a disease or condition wherein damage to the brain is associated with or mediated by astroglial activation or astrogliosis (e.g., Alzheimer's disease, frontotemporal dementia (FTD), and other tauopathies and dementias. In particular, the conditions which a subject in need suffers from or is suspected of suffering from include, but are not limited to traumatic brain injury (TBI), chronic traumatic encephalopathy (CTE), Alzheimer's disease (AD), and frontotemporal dementia (FTD).

As used herein, the term “brain injury” includes traumatic injuries and injuries as a result of disease, in particular neurodegenerative diseases and dementias. Thus, “brain injury” includes, but is not limited to mild, moderate, or severe trauma to the brain such as that received in military conflict, sports injury, accidents and falls, and the like, and also includes but is not limited to injury to the brain as a result of any tauopathy or dementia. In a specific embodiment, the brain injury is accompanied by, associated with, or mediated by astrogliosis or astroglial activation. Types of traumatic brain injury include closed or open head injuries, CTE, for example. Types of non-traumatic brain injury include tauopathy (a neurodegenerative disease associated with accumulation of Tau protein in neurofibrillary or gliofibrillary tangles in the brain, e.g., Alzheimer's disease, primary age-related tauopathy, CTE, frontotemporal dementia, Creutzfeldt-Jakob disease, forms of parkinsonianism, certain brain tumors, and the like).

As used herein, the term “astrogliosis,” also referred to as “astrocytosis,” “astroglial activation,” or “reactive astrocytosis,” refers to an increase in the number of astrocytes after destruction of neurons due to trauma, infection, ischemia, stroke, immune responses, neurodegenerative disease, or any cause. Astrogliosis also is accompanied by changes in astrocyte morphology and function.

As used herein, the term “GFAP” refers to intact glial fibrillary acidic protein, an intermediate filament protein encoded by the GFAP gene in humans and expressed in the central nervous system, primarily in astrocytes. All isoforms of the GFAP protein are included in this definition. As used herein, the term also refers to breakdown products of GFAP, including natural and synthetic peptides derived from the sequence of GFAP. Therefore, “GFAP or a fragment thereof” refers to full length GFAP isoforms or any breakdown product, for example, the central core breakdown product GFAP-38K (with residue range about 79-383 in GFAP-α), the N-terminal head region with residue range about 1-72 in GFAP-α, and the C-terminal tail region with residue range about 378-432 in GFAP-α, i.e., the truncated forms of GFAP with apparent molecular weights of about 44 kDa, 42 kDa, 40 kDa and 38 kDa.

As used herein, the term “immunization” refers to any passive or active method of introducing or producing antibodies specific to a particular antigen. For example, immunization for GFAP includes administration of antibodies that specifically recognize GFAP or an epitope or hapten of GFAP to a subject, or an aptamer that binds to GFAP; such types of immunization relate to a passive immunization. Immunization also includes administration of GFAP protein or a peptide derived from GFAP to the subject in order to stimulate the immune system of the subject to produce antibodies that specifically recognize GFAP, an active immunization. Both active and passive immunization is included in the term “immunization” and all of its cognates, unless stated otherwise.

As used herein, the term “GFAP antibody (“anti-GFAP antibody”) or a fragment thereof” refers to an intact anti-GFAP antibody or a combination of fragmented heavy and light chains of immunoglobulin or single chain fusion protein containing heavy-light chain plus light brain variable fragments. Any type of antibody is included within the term if it specifically binds to GFAP or a fragment or breakdown product of GFAP. As used herein, the term “GFAP aptamer” refers to one or more single-stranded oligonucleotide (DNA or RNA) molecules that bind to a specific target molecule, e.g., GFAP or a fragment thereof.

As used herein, the term “therapeutically effective amount” refers to an amount of a compound or composition that, when administered to a subject for treating a disease or disorder, or at least one of the clinical symptoms of a disease or disorder, is sufficient to affect such disease, disorder, or symptom. A “therapeutically effective amount” includes an amount that ameliorates, reduces or cures the disease, disorder, or symptom and may vary depending, for example, on the compound, the disease, disorder, and/or symptoms of the disease or disorder, severity of the disease, disorder, and/or symptoms of the disease or disorder, the age, weight, and/or health of the subject to be treated, the capacity of the individual's immune system to synthesize antibodies, the degree of protection desired, the formulation of the vaccine, the treating doctor's assessment of the medical situation, and other relevant factors. A therapeutically effective amount can be a single dose or a series of doses administered to a subject in need thereof. An appropriate amount in any given instance may be readily ascertained by those skilled in the art or can be determined by routine experimentation.

### 2. Overview

Glial fibrillary acidic protein (GFAP) is a biomarker candidate for TBI diagnosis and prognosis, but also a pathological hallmark involved in TBI pathology. In some TBI patients, there also is a blood-based autoantibody response to GFAP proteins. In this invention, the toxic form of GFAP protein was evaluated by passive immunotherapy (anti-GFAP antibody treatment) in a mouse model of TBI. Current biomarker candidates, including GFAP, neuronal-specific enolase (NSE), the phosphorylated axonal form of the heavy neurofilament (pNF-H), neurodegeneration-linked microtubule associated protein Tau and its phosphorylated form (P-Tau), as well as behavioral changes were measured after therapy. Results showed that immunization with GFAP protein attenuated the increased serum levels of GFAP at 20 days post brain injury and reduced the serum levels of pNF-H, Tau or P-Tau at 50 days following TBI. Pre-immunization also reduced the overexpression of GFAP, Tau and P-Tau in brain cortex tissues. Treatment with GFAP immunization alleviated anxious behavior at days 10 and 20 following brain injury and improved cognitive performance at day 20 post injury. These findings indicate that active GFAP immunization by either GFAP protein or peptides or passive immunization with anti-GFAP antibody treatment has a therapeutic value in suppressing astroglial activation/astrogliosis, and in treating brain injures such as Alzheimer's disease (AD) and chronic traumatic encephalopathy (CTE).

### 3. Description of Embodiments of the Invention

Glial fibrillary acidic protein (GFAP) is a structural protein unique to astrocytes. GFAP is a component in the cytoskeletal structure of astroglial cells and operates in maintaining their mechanical strength, as well as supporting neighboring neurons and the blood-brain barrier (BBB). Because GFAP is enriched in astroglial cells in the CNS, it can be used as a biomarker for diagnosis or prognosis of TBI. Shortly following TBI, there is a release of high concentration of GFAP (intact protein, 50 kDa) and its fragments (peptides, also known as breakdown products (BDPs), 38 kDa-44 kDa) from astrocytes into the extracellular fluid and cerebrospinal fluid and blood.

Therefore, GFAP is a pathological hallmark of astrogliosis in TBI pathology. An increase in GFAP is believed to be an indicator of the astroglial activation and hypertrophy observed following brain injury. Activated astrocytes are known to mediate the neuroinflammation process, including the release for proinflammatory cytokines (e.g. IL-6, TNF-alpha). Activated astroglia cells also form the so-called glial scar that can further inhibit neuroregeneration. After TBI and rupture of the BBB, GFAP is released from damaged astrocytes, enters the bloodstream where it can trigger an immune response in a subset of TBI patients. Therefore, in some TBI patients, there is a blood-based dominant autoantibody response to GFAP protein apparent after injury. Currently, it is not known if astroglial cell activation is beneficial or detrimental to recovery from TBI, however it may be both. Neuroinflammation initially can be beneficial by removing cell and neurotoxic debris from the site of injury, but sustained and unresolved neuroinflammation can be harmful.

The immune system has both detrimental and beneficial effects on the nervous system under stress or challenges. Multiple sclerosis is a typical example for an abnormal immune disease that involves a central nervous system antigen. There is some evidence that TBI, whether mild or severe, has a high risk of triggering autoimmunity with the release of brain-specific proteins (MBP, S100B and glutamate receptors) into the peripheral blood system and that there is a correlation of serum anti-S100B and white matter disruption. A dominant anti-GFAP autoantibody response occurs within 5-10 days in a subset of patients with severe TBI and a persistent upregulation of this response is present in the subacute to chronic phase after TBI, as well as after repeated TBI insults. Studies have shown a correlation between certain brain injury and the autoantibodies levels. However, there is still no direct evidence as to whether the autoantibodies will further exacerbate the damage or such immunological responses may benefit the outcome.

Disease-modifying immunotherapies for acute brain injuries such as TBI and degeneration such as AD were evaluated here. A number of neuropathy biomarkers, including GFAP, neuronal-specific enolase (NSE), phosphorylated axonal form of the heavy neurofilament (pNF-H), axonally located microtubule associated protein, Tau, and its phosphorylated form (p-Tau), were measured in brain tissue and/or biofluid after brain injury, with and without GFAP immunotherapy, in a mouse model of TBI. Behavioral tests as well as histological analysis also were performed to address the effects of GFAP immunotherapy.

Mice were pre-immunized with GFAP protein to achieve a robust anti-GFAP IgG titer as monitored by ELISA, before TBI surgery and studied to determine the effects of anti-GFAP immunotherapy. Both active and passive vaccines were tested. Active immunization involves administering a pathogenic agent (antigen) to elicit an immune response and production of antibodies directed to the antigen. Passive immunization involves administering a specific antibody that targets a given antigen.

Given the growing evidence that TBI, even mild TBI, has a high risk of triggering autoimmunity with the release of brain-specific proteins (such as MBP, S100B and glutamate receptors) into the peripheral blood system, efficacious treatments for TBI become more important. The dominant response in some patients with TBI is an anti-GFAP autoantibody response within 5-10 days in severe TBI and a persistent upregulation of this response in the subacute to chronic phase after TBI, or after repeated TBI insults. However, there is no direct evidence indicating whether the autoantibodies further exacerbate the damage or whether such immunological responses benefit the outcome of TBI; the immune system has both detrimental and beneficial effects on the nervous system under stress or challenges.

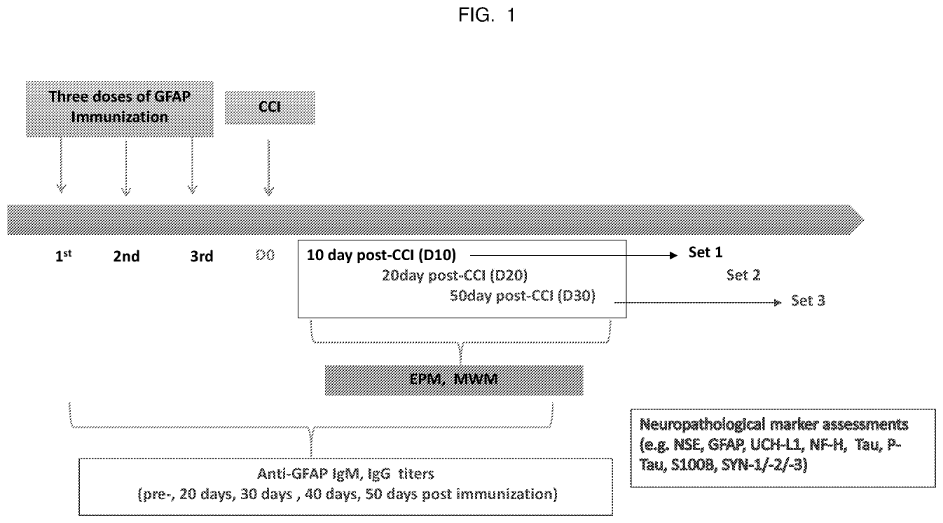

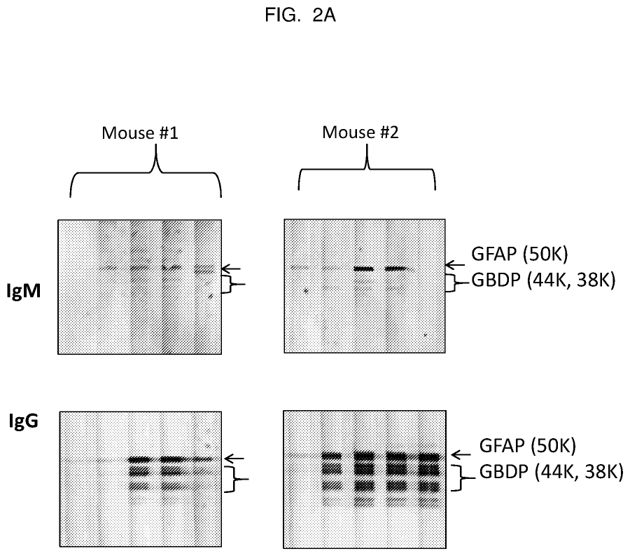

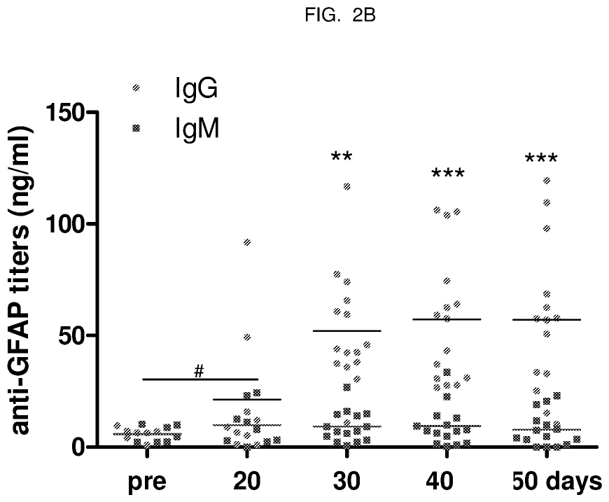

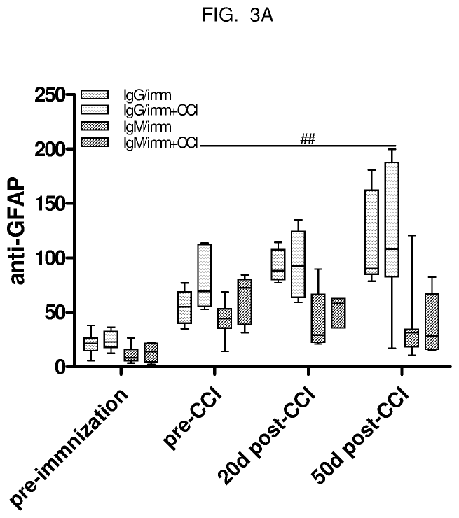

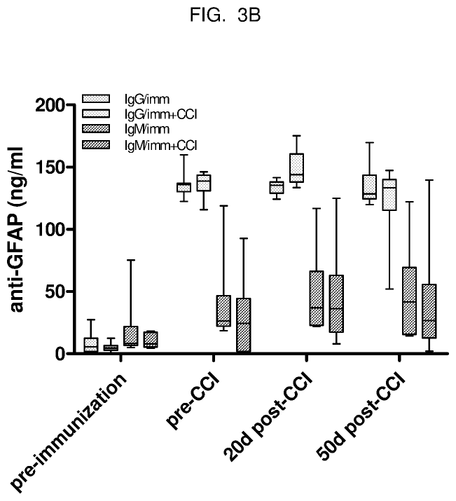

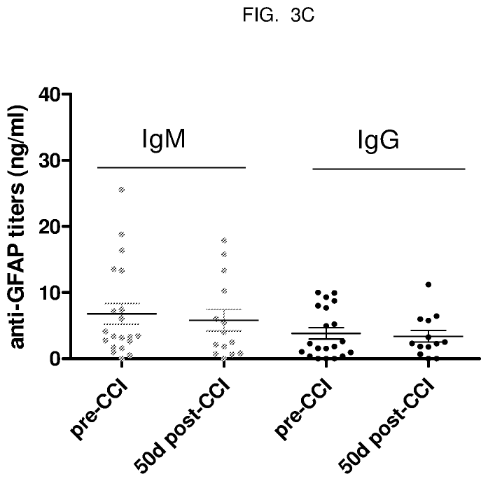

In this study, pre-immunization was able to amplify innate autoimmunity after TBI, allowing an examination of the effect of this immune response. First, mice received a 3-dose series of GFAP protein 14 days apart. Since pre-immunization could amplify innate autoimmunity after TBI, allowing examination of the effect of the immune response, mice were studied using the following basic protocol. See FIG. 1. First, the mice received 3-dose series of immunizations with GFAP protein, 14 days apart. After the third dose, the mice immediately received CCI surgery. Thirty days after the initial immunization, the anti-GFAP IgG titers in serum reached a peak level and this high level was maintain during the following 20 days. See FIG. 2. Although no further booster immunizations were given to the mice, the anti-GFAP IgG titer was sustained for at least 50 days post-injury using immunoblot measurements. See FIG. 3A. Unlike human TBI patients, brain injury in the mice did not trigger additional autoimmune response. This indicates that these beneficial results were likely due to the pre-immunization with GFAP antigen only. Based on this finding, in the following experiments, we aimed to evaluate the mechanistic effects of GFAP pre-immunization.

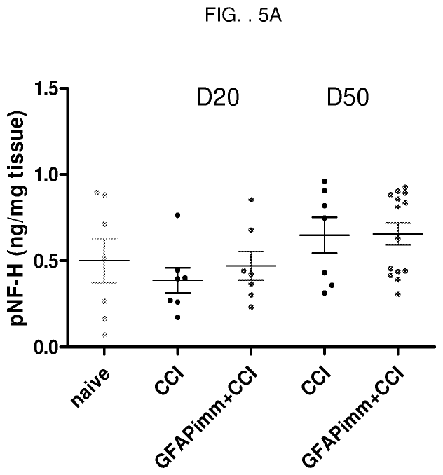

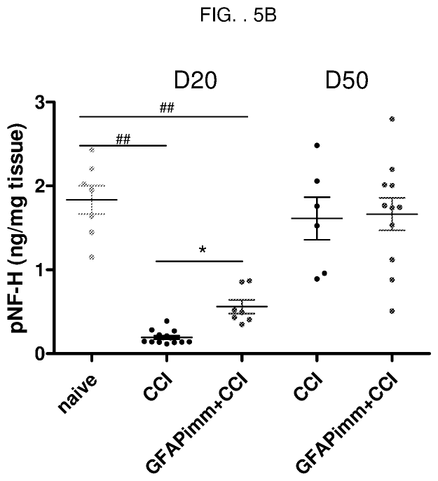

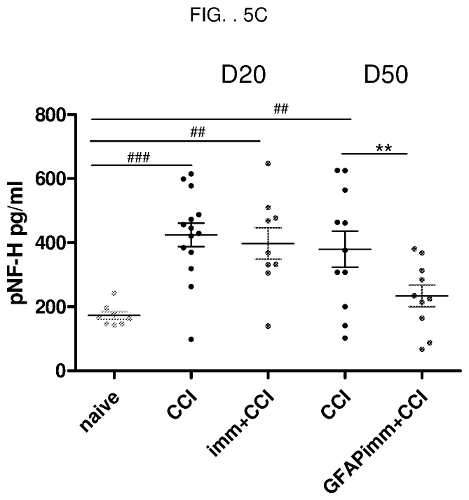

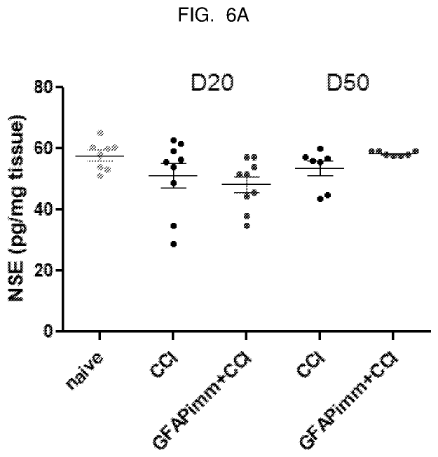

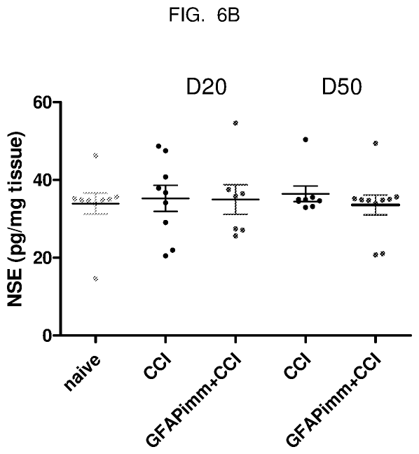

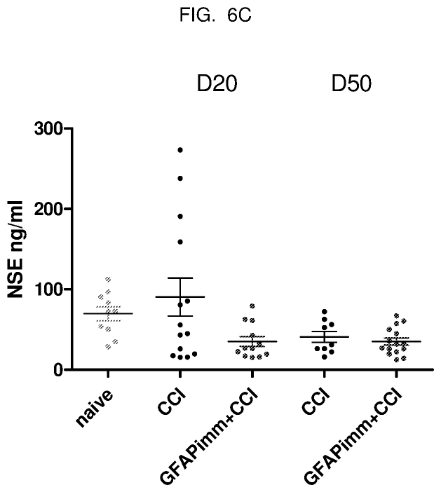

GFAP, pNF-H, NSE, Tau and P-Tau indicate the molecular and biochemical changes induced by TBI. The levels of these proteins were measured in serum as well as brain tissues (cortex and hippocampus) at a chronic phase (Day 20 and Day 50 post-TBI). Evidence showed that biofluid (CSF, blood) levels of most acute TBI markers will return to baseline levels within a matter of days following TBI, especially for those who suffered from mild brain injury. However, subacute and chronic effects of TBI can persist for months following the initial injury event. NSE is an acute marker which can reach a peak level within few hours. Thus, there would be no detectable change at either Day 20 or Day 50 following TBI here. See FIG. 6.

GFAP is an acute/subacute marker that increase immediately after TBI and then climbs to a peak level a few hours after TBI, but takes longer to return to baseline. Here, GFAP pre-immunization had the beneficial effect of reducing elevated GFAP levels serum at Day 20, indicating reduced injury from the TBI. GFAP pre-immunization also suppressed GFAP levels in the injured cortex at Day 20 post-injury. These results were confirmed by immunohistochemical staining for GFAP in brain. Taken together, the GFAP pre-injury immunization therapy achieved the goal of reducing elevated GFAP levels in brain tissue and in circulating blood.

pNF-H is a delayed axonal injury marker. After TBI, pNF-H levels at Day 20 were reduced in hippocampus, suggestive of delayed axonal degeneration. Furthermore, at both Day 20 and Day 50, there were increases in released pNF-H levels in serum, suggesting the proteins were released from damaged cells into the peripheral blood system. Importantly, pre-immunization with GFAP partially restored hippocampal pNFH-H levels at Day 20, showing a decrease in pNF-H release, hence cell damage. At the same time, immunization treatment also significantly attenuated the released pNF-H levels at Day 50. See FIG. 5. Thus, GFAP immunotherapy appears to have protective effects against delayed axonal injury.

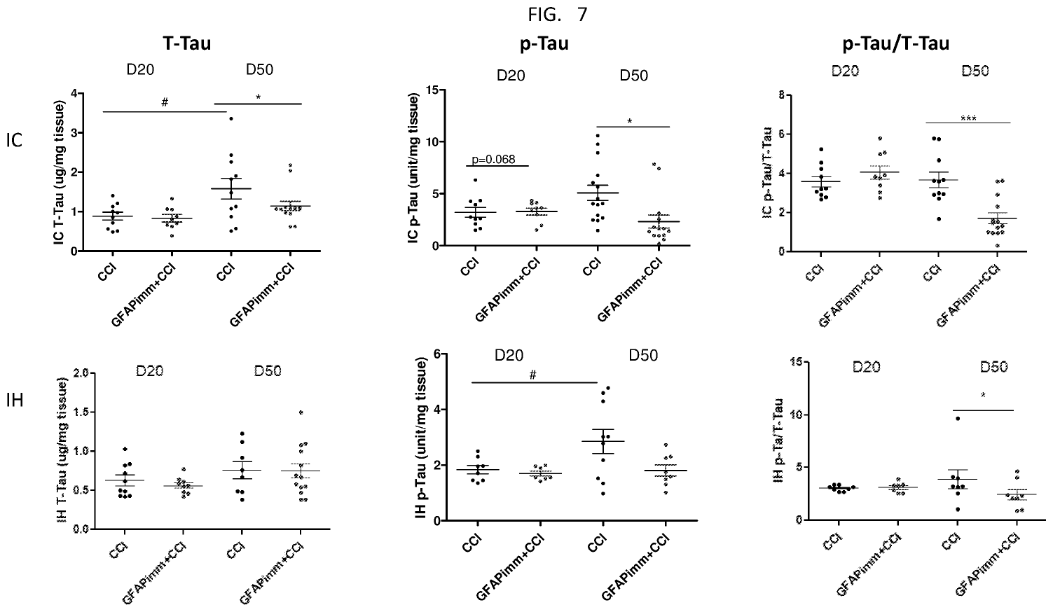

Tau plays a pivotal role in the pathogenesis of neurodegenerative disorders. Hyperphosphorylated Tau (P-Tau) aggregates of tau, forming neurofibrillary tangles (NFTs), constitute a pathological hallmark of Alzheimer disease (AD) and fronto-temporal dementia (FTD) and PD. Tau suppression in a neurodegenerative mouse model improves memory function and stabilized neuron numbers. Tau and P-Tau or P-Tau/T-Tau ratio also are considered chronic TBI biomarkers relating to neurodegeneration. Here, chronic tauopathy after TBI, with a higher total-tau or P-tau expression in either cortex or hippocampus tissues at Day 50 compared to that at Day 20 was found. See FIG. 7. GFAP immunization reduced the PTau/T-Tau ratio in injured cortex and injured hippocampus at Day 50 post-injury. See FIG. 7. Serum Tau and P-Tau were not examined since currently there still are limitations to robust detection of Tau and P-Tau levels in rodent serum. Also, the P-Tau concentration is about 2-5% of total Tau (data not shown). Thus, more sensitive methods are required for P-Tau assay in rodent serum samples. Overall, GFAP pre-immunization showed beneficial effects after TBI, demonstrated by several TBI biomarkers, which indicates a clinical use for the treatment. Importantly, the ability of GFAP immunization to attenuate tauopathy (increased Tau and P-Tau levels in brain and biofluids) demonstrates that such immunization treatment can attenuate neurodegenerative conditions with a tauopathy component, such as CTE, AD, PD and FTD.

A TBI can cause chronic effects, including CNS and systemic sequelae such as cognitive impairment (memory and executive dysfunction), neurological symptoms (headache, sleep disturbance, and pain), neuro-endocrine dysfunction, and mental health impairment (depression, anxiety, apathy, and suicidality). Here, pre-immunization with GFAP attenuated the chronic neurological symptoms following TBI, cognitive impairment and anxiety. These were measured using classic techniques, the Morris water maze (MWM) and the elevated plus maze (EPM) at 10 days, 20 days and 50 days after brain injury. Treatment with GFAP immunization alleviated anxious behavior at Day 10 and Day 20 following brain injury. Pre-immunization with GFAP also improved impaired MWM performance at Day 20 and it showed a strong similar beneficial trend at Day 10. However, it did not improve either of these neurological deficits at Day 50. The serum anti-IgG titer in some mice was reduced at Day 50 post injury although the group average titer still kept a high level. The mice with higher titers showed a good performance in both the MWM and EPM tests, while those with lower titers showed poorer performance (individual mouse data not shown).

While GFAP immunization produced improvement in functional outcomes, it did not significantly reduce lesion volume. Without wishing to be bound by theory, it may be possible that GFAP immunization might be exerting some of its effects by promoting post-injury neuroplasticity and neuroregeneration. Because anti-GFAP IgG or IgM titer dropped in some mice at Day 50 after the initial immunization (see FIG. 2A), additional boosts might be necessary to maintain optimal effects of the immunotherapy. Thus, the immunization strategies and titer of antibody would influence the effectiveness of the immunotherapy efficacy. Preferably, a higher and more sustained level of GFAP antibody is maintained throughout the treatment period for maximum benefits of the therapy.

In conclusion, disease-modifying GFAP-directing immunotherapies or aptamer-based therapy are possible treatments for diseases that involve neural tissue damage or neural repair, including but not limited to acute traumatic brain injury (e.g., TBI, CTE and the like), spinal cord injury, and chronic neurodegenerative brain damage (e.g., AD, PD, MS, FTD and other dementias). The stated GFAP-directed treatments also can include other neural diseases or neurological disorders such as stroke (ischemic and hemorrhagic), glioblastoma, vanishing white matter disease, and brain hemorrhage (intracerebral hemorrhage, subarachnoid hemorrhage), epilepsy, Huntington's disease (HD), amyotrophic lateral sclerosis (ALS), hypoxic ischemic encephalopathy (HIE), neural damage due to drug or alcohol use or abuse (e.g., from amphetamines, ecstasy/MDMA, or ethanol), prion-related disease, peripheral neuropathy, diabetic neuropathy, and chemotherapy-induced neuropathy and neuropathic pain. This invention provides an astroglia protein-targeting immunotherapy in a mouse model of TBI and shows the beneficial effects of GFAP immunization on reducing TBI pathological biomarker signature as well as improving behavioral outcome. These findings offer valuable implications regarding our understanding of GFAP as a drug target.

The brain injuries contemplated for use with the invention include any disease or condition involving damage to the brain in which astroglial activation, astrogliosis, or both are involved in the pathologic processes or as biomarkers of the condition. Astrogliosis is a pathologic abnormal increase in the number of astrocytes after destruction of nearby neurons due to trauma, infection, ischemia, autoimmune responses, or neurodegenerative disease such as Alzheimer's disease. Astroglial activation (reactive astrocytes) is a related phenomenon where the astrocytes in the area of an injury undergo changes in molecular expression and morphology as a response to physical or metabolic insult such as infection, ischemia, immune responses, inflammation, hemorrhage, trauma and the like. These cells can protect neurons by taking up toxins from the area and repairing the blood brain barrier, but also can have negative effects that prevent axon regeneration and produce scar tissue.

Brain injuries that can be treated according to the invention include any brain injury that is mediated by astrogliosis/astroglial activation, or that is accompanies by astrogliosis/astroglial activation. These injuries include but are not limited to TBI, stroke (ischemic and hemorrhagic), SCI, brain hemorrhage (for example intracerebral hemorrhage and subarachnoid hemorrhage), CTE, AD, FTD, PD, MS, and ALS.

TBI occurs due to physical trauma to the brain, including closed head injury and penetrating head injury. Typically, TBI occurs due to a fall, vehicle collision, work injury, sports injury, violence, and the like. TBI can result in various physical, cognitive and behavioral symptoms, depending on the area of the brain affected and its severity, symptoms which may be permanent. It is a major cause of death and disability. Current treatment focusses on minimizing the damage caused, and prevention.

CTE, also referred to as traumatic encephalopathy syndrome or dementia pugilisitca, is a neurodegenerative condition caused by repeated head injuries, and tends to get worse over time, resulting in dementia. The cause frequently is repeated injury in contact sports, the military, domestic violence, or repeated banging of the head. Firm diagnosis often is made only at autopsy, and no treatment is available and focusses on maintenance and support only.

Neurodegenerative diseases are those which involve progressive loss of neurons or their function, including death of neurons, and which result in a progressive loss of brain function. Neurodegenerative diseases associated with a tauopathy (a pathological aggregation of Tau protein in neurofibrillary or gliofibrillary tangles in the brain) include Alzheimer's disease, FTE, and the like. CTE also sometimes also is classified as this type of neurodegenerative disease. Neuroinjury and neurodegenerative diseases and conditions associated with astrogliosis and/or astroglial activation include TBI, stroke (ischemic and hemorrhagic), SCI, brain hemorrhage (including intracerebral hemorrhage, subarachnoid hemorrhage), CTE, AD, FTD, PD, HD, MS and ALS.

The methods of the invention involve immunization for GFAP either by passive or active means. Active immunization involves administration of a GFAP antigen in order to induce an immune response which includes production of anti-GFAP antibodies, i.e., antibodies that specifically recognize one or more epitopes on GFAP. The GFAP antigen can include the intact protein, or peptide derivatives of the intact sequence. Preferred antigens for active immunization are full length GFAP isoforms, 11 length GFAP-α (residue 1-432), central core GFAP-breakdown product (38 kDa; with residue range about 79-383 in GFAP-α), N-terminal head region of GFAP-α (with residue range about 1-72 in GFAP-α), and C-terminal tail region of GFAP-α (with residue range about 378-432 in GFAP-α). Intact GFAP protein can be used, or any peptide derived from the intact protein sequence. Alternatively, the antigen can be prepared using a short peptide covalently attached to a larger protein to serve as a hapten.

Active immunization can involve a single dose administration of GFAP antigen or multiple doses administered over a period of time. For example, administrations of GFAP antigen can be administered daily, weekly, every two weeks, monthly, every two months or at any convenient interval as determined by the practitioner. The dose of the antigen will depend on the condition of the subject to be treated and the subject's immune system, and can be any amount from about 1 mg/kg to about 200 mg/kg, preferably about 5 mg/kg to about 150 mg/kg, more preferably about 10 mg/kg to about 100 mg/kg, and most preferably about 20 mg/kg to about 75 mg/kg. When intact protein is the antigen, the dose per administration generally is about 1 mg/kg to about 200 mg/kg, preferably about 20 mg/kg to about 75 mg/kg. After an initial administration, booster administrations of antigen optionally can be given. These booster doses can be the same amount and antigen as that administered in the initial administration, or can be a smaller dose.

The antigen preferably is administered in the form of a pharmaceutical composition or vaccine composition that contains the antigen and a pharmaceutically acceptable carrier, optionally including an adjuvant to stimulate the subject's immune response to the antigen. Preferred administration is by injection, which can include intramuscular, subcutaneous, intradermal, intraperitoneal, intravenous, intra-arterial, intrathecal, local injection to the area of injury, or any convenient injection route. Alternatively, the administration can be nasal, oral, or any suitable or convenient route of administration.

Passive immunization (passive antibody therapy) can be more practical and predictable than active immunization since active immunization relies on an individual's immune response to the injected antigen. Therefore, the invention also relates to methods of passive immunization for GFAP (anti-GFAP antibody therapy). Passive immunization involves administration of antibodies directly to the subject. For passive immunization (anti-GFAP antibody-based therapy), the preferred antigen for producing the therapeutic antibody is one of the full length GFAP isoforms, full length GFAP-α (residue 1-432), central core GFAP-breakdown product (GBDP) of 38K (with residue range about 79-383 in GFAP-α), N-terminal head region with residue range about 1-72 in GFAP-α, or C-terminal tail region with residue range about 378-432 in GFAP-α.

The antibodies administered can include polyclonal or monoclonal antibodies, preferably monoclonal antibodies or recombinant antibodies. Bispecific antibodies can be used, as well as antibody fragments, or single chain fusions of heavy and light chain variable regions, so long as they exhibit the desired biological activity, i.e., specific recognition of and binding to GFAP protein or peptide. The desired biological activity of the anti-GFAP antibodies include specific and high affinity binding (dissociation constant of <10⁻⁶) to the full length protein of one or more of the GFAP isoforms, their breakdown products (GBDP) (such as GBDP-38K) or fragments, and C- and N-terminal regions.

Any of the five major classes of antibodies can be used for passive immunization, including IgA, IgD, IgE, IgG, and IgM, as well as any of the subclasses (isotypes), e.g., IgG1, IgG2, IgG3, IgG4, IgA1, and IgA2. Intact or whole, native, antibodies can be used, as well as antibody fragments. Examples of antibody fragments include Fab, Fab′, F(ab′)2, and Fv fragments; single chain Fv fragments; diabodies; linear antibodies; single-chain antibody molecules; and multispecific antibodies formed from antibody fragments as are known in the art.

The term “monoclonal antibody” as used herein refers to an antibody obtained from a population of substantially homogeneous antibodies, i.e., the individual antibodies comprising the population are identical except for possible mutations, e.g., naturally occurring mutations, that may be present in minor amounts. Thus, the modifier “monoclonal” indicates the character of the antibody as not being a mixture of discrete antibodies. Monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity.

“Humanized” antibodies also are contemplated for use with the invention. Such humanized antibodies are known in the art and are chimeric antibodies that contain minimal sequence derived from non-human immunoglobulin. In general, the humanized antibody will comprise substantially all of at least one, and typically two, variable domains, in which all or substantially all of the hypervariable loops correspond to those of a non-human immunoglobulin (i.e., mouse) but the remainder of the antibody contains human sequences that are less likely to trigger an immune response on their own. Human antibodies, i.e., antibodies produced in or by a human and containing only human sequences, or synthetically produced using various techniques known in the art, including phage-display libraries also are contemplated for use with the invention.

Aptamers also are contemplated for use with the invention. Aptamers are single strand oligonucleotide (DNA or RNA) molecules that bind to a specific target molecule. Importantly, it has been demonstrated that aptamers often can substitute for antibody as therapeutic or diagnostic agent for engaging target molecules such as proteins with high affinity. See reference 75, below. Thus, in this case, GFAP-binding aptamers can be administrated to human as a GFAP- and astrogliopsis-targeting treatment for neuroinjury, neurodegeneration, neuro-disease and neuro-repair where GFAP and astrogliosis is involved.

Passive immunization can involve a single administration of GFAP antibodies, treatment with GFAP-binding aptamers, or multiple doses of either or both, administered over a period of time. For example, administrations of GFAP antibodies can be administered daily, weekly, every two weeks, monthly, every two months or at any convenient interval as determined by the practitioner. The dose of the antibodies will depend on the condition of the subject to be treated and the subject's immune system, and can be any amount from about 1 mg/kg to about 200 mg/kg, preferably about 5 mg/kg to about 150 mg/kg, more preferably about 10 mg/kg to about 100 mg/kg, and most preferably about 20 mg/kg to about 75 mg/kg. After an initial administration, booster or repeated administrations of antibody optionally can be given. These booster or repeated doses can be the same amount and antibodies as that administered in the initial administration, or can be a smaller dose, and can be given at any suitable interval, for example daily, weekly, bi-weekly, or monthly.

The antibodies or aptamers preferably are administered in the form of a pharmaceutical composition or vaccine composition that contains the antibodies and a pharmaceutically acceptable carrier. Preferred administration is by intravenous injection, but also can include intramuscular, subcutaneous, intradermal, intraperitoneal, intra-arterial, intrathecal, local injection to the area of injury, or any convenient injection route. Alternatively, the administration can be nasal, oral, or any suitable or convenient route of administration.

Pharmaceutical compositions and vaccine compositions preferably contain a pharmaceutically acceptable carrier or vehicle. The terms “pharmaceutically acceptable carrier,” “pharmaceutically acceptable excipient,” or “pharmaceutically acceptable vehicle” refer to any convenient compound or group of compounds that is not toxic and that does not destroy or significantly diminish the pharmacological activity of the therapeutic agent with which it is formulated. Such pharmaceutically acceptable carriers or vehicles encompass any of the standard pharmaceutically accepted solid, liquid, or gaseous carriers known in the art, such as those discussed in the art.

Suitable carriers depend on the route of administration contemplated for the pharmaceutical composition. Such routes can be any route which the practitioner deems to be most effective or convenient using considerations such as the patient, the patient's general condition, and the specific condition to be treated. For example, routes of administration can include, but are not limited to: oral, intravenous, intra-arterial, intrathecal, subcutaneous, intraperitoneal, rectal, vaginal, topical, nasal, local injection, buccal, transdermal, sublingual, inhalation, transmucosal, wound covering, and the like. Therefore, the forms which the pharmaceutical composition can take will include, but are not limited to: tablets, capsules, caplets, lozenges, dragees, pills, oral solutions, sterile powders for dilution, powders for inhalation, vapors, gases, granules, sterile solutions for injection, transdermal patches, buccal patches, inserts and implants, rectal suppositories, vaginal suppositories, creams, lotions, ointments, topical coverings, and the like, and can include suitable containers such as vials, ampules, bottles, pre-filled syringes and the like. Preferably, administration is by injection, therefore preferred forms for the pharmaceutical compositions and vaccine compositions include solutions for injection, suspensions, powders or granules for dilution, pre-filled syringes, and the like, or any suitable or convenient form.

The preferred vehicles, carriers, and/or excipients include solvents, fillers, diluents, pH adjusters, salts, sugars, preservatives, antioxidants, colorings, suspending agents, chelating agents, surfactants, buffers, and the like. Examples of such preferred excipients include solvents (water, saline solution, buffered saline solution, glycerol, and the like), salts (e.g., sodium, potassium, chloride, phosphate, carbonate, citrate, and the like), and sugars (e.g., lactose, sucrose, and the like).

Preferred excipients in an active vaccine or immunization composition also include one or more adjuvant. Suitable adjuvants include, but are not limited to aluminum hydroxide, oils such as paraffin oil or food oils, adjuvants (e.g., Freund's incomplete adjuvant, Freund's complete adjuvant, or any suitable adjuvant, or any pharmacological or immunological agent that modifies the immune response to result in a higher amount of antibodies specific to the antigen administrated. Other additives known to the person of skill also can be used.

In summary, it has now been found that the presence of antibodies to GFAP, a signaling molecule coinciding with astroglial activation can have beneficial effects on the outcome of brain injury of this type. The invention therefore is a method of treatment of brain injury that involves astrogliosis or astroglial activation by administering a passive or active immunization (vaccine) of GFAP.

### 4. Examples

It is to be understood that this invention is not limited to the particular processes, compositions, or methodologies described, as these may vary. It is also to be understood that the terminology used in the description is for the purpose of describing the particular versions or embodiments only, and is not intended to limit the scope of the present invention which will be limited only by the appended claims. Unless defined, otherwise, all technical and scientific terms used herein have the same meanings as commonly understood by one of ordinary skill in the art. Although any methods and materials similar or equivalent to those described herein can be used in the practice or testing of embodiments of the present invention, the preferred methods, devices, and materials are now described. All publications mentioned herein, are incorporated by reference in their entirety. Nothing herein is to be construed as an admission that the invention is not entitled to antedate such disclosure by virtue of prior invention.

### Example 1. General Methods

A. Animal Care

Both female and male C57/BL6 mice (6-8 weeks) were used for all experiments. Mice received first immunization at 6-8 weeks and receive cortical control impact (CCI) surgeries on 10-12 weeks as a model of traumatic brain injury. For biomarker assays, female and male mice were included, however only male mice were enrolled in behavioral examinations. Mice were housed in a temperature-controlled room (22° C.) with a 12-hour light/dark cycle. All animals had access to food and water ad libitum.

B. Immunizations

Mice were randomly assigned and evenly distributed into three treatment groups: naive, CCI, or CCI plus immunization with GFAP (GFAPimm+CCI). Each group contained 15-20 mice. For behavioral tests, at least 10 male mice were included in each group. Mice in the GFAPimm+CCI group were dosed every two weeks (for a total of three doses) by subcutaneously injecting with 25 μg GFAP mixed with incomplete Freund's adjuvant. The day on which CCI was performed was considered Day 0. Following CCI, mice were monitored closely each day for signs of infection, bleeding, and general distress until the main study concluded on certain days post injury.

C. Animal TBI Model:

Following a 28-day immunization, TBI was induced by CCI using the Leica Impact™ One system (Leica Biosystems™, Buffalo Grove, Ill.) as described in the art. See Yang et al., J. Cereb. Blood Flow Metab. 34:1444-1452 (2014). Briefly, mice were anesthetized using an isoflurane vaporizer and monitored throughout the procedure. A heating pad was used and monitored during surgery and maintained at 39° C. The core body temperature of the mouse was continuously monitored by a rectal thermistor probe and maintained at 37±1° C. A midline incision approximately 1 cm in length was made along the head and the skin was pulled aside using small bulldog clamps. With the skull exposed, a dental scraper was used to partially remove the fascia in order to better visualize anatomical markers. The dura mater was kept intact over the cortex. Bregma was located, and a concave 22-gauge stainless steel disk, 4 mm in diameter, was affixed to the skull using tissue adhesive just caudal to this point. Animals were then placed into a stereotaxic frame (Lecia Impact™ One, Leica Microsystems™, Inc.) and the head was secured to prevent movement during impact. The arm of the impactor was then positioned such that the impactor probe (mm diameter) was directly centered over the metal disk. Brain trauma was produced by impacting the right cortex (ipsilateral cortex) with a 4 mm diameter impactor tip at a velocity of 3.5 m/s, 1.5 mm compression depth and a 200 milliseconds dwell time. After impact, animals were monitored and returned to their home cage once they became fully ambulatory.

D. Serum and Tissue Sample Preparation

Subsequent to euthanasia, mouse cortex and hippocampus were isolated from the brain and pulverized to a fine powder with a mortar and pestle set over dry ice. The pulverized brain tissue then was lysed for 2 hours at 4° C. in 20 mM Tris-HCl pH 7.4, 5 mM EDTA, 5 mM EGTA, 1% Triton X-100, and 1 mM DTT, and complete protease inhibitor cocktail (Roche™), followed by centrifugation at 10,000×g for 10 minutes at 4° C. Serum samples were obtain from the anesthetized rats by cardiac puncture followed by centrifugation at 1000×g for 5 minutes after the blood had clotted. The supernatant then was transferred into new Eppendorf™ tubes. Both brain tissue and centrifuged blood samples were snap-frozen on dry ice and stored at −80° C. for further analysis.

E. Manifold Autoantibody Immunoblotting Assay

Samples were subjected to SDS-PAGE on 4-20% Tris-glycine gels and electrotransferred to polyvinylidene fluoride (PVDF) membrane. PVDF membranes then were clamped into the Mini-Protean™ II Multiscreen apparatus (Bio-Rad™), and individual lanes were blocked and probed with mouse serum diluted at 1:100. Secondary antibodies for detection were either alkaline phosphatase (AP)-conjugated goat anti-mouse IgG+IgM or AP-conjugated donkey anti-mouse IgG, diluted 1:10,000 (Jackson ImmunoResearch™). Quantitation of autoantibody reactivity on immunoblots was performed via computer-assisted densitometric scanning (Epson™ 8836 XL high-resolution scanner and NIH Image J™ densitometry software). Autoantibody levels were expressed in arbitrary densitometry units.

F. Anti-GFAP IgG/IgM Enzyme-Linked Immunosorbent Assay (ELISA)

Ninety-six-well ELISA plates were coated with recombinant human GFAP protein (Dx-SYS™) or human brain extract, 1 μg/well). After plate preparation, 1 μL of mouse sample was mixed with 99 μL of Start-Block™ buffer (Fisher™) and then transferred to each well (1:100 dilution) with incubation at 4° C. overnight with shaking. Plates were washed again 4 times with Tris-Buffered Saline and Tween™ 20 (TBST) wash buffer. Anti-mouse IgG/IgM HRP-conjugate (Jackson ImmunoResearch™, diluted 1:10,000 in TBST Start-Block™ blocking buffer) was added as a 100 μL aliquot to each well. Plates were incubated at 25° C., with shaking for 45 minutes. After plate washing 4 times with TBST, 100 μL TMB substrate was added to develop color for 15 minutes. Stop Solution (100 μL) then was added, and plates were read at 450 nm for the yellow color of the final product. Standard curves were produced by adding 0, 17, 26, 39, 58.5, 88, 131.5, 198, 296, 444, 666 and 1,000 ng/mL (50 uL) of either purified human IgG or human IgM (Sigma™). Upon blocking and washing as above, anti-mouse IgG or IgM HRP-conjugate (1:10,000 in TBST Start-Block™ blocking buffer) was added, followed by TMB substrate. OD readings as a result of the presence of anti GFAP IgG or IgM were converted to IgG or IgM concentration in μg/mL.

G. TBI Neuropathological Biomarker Assessments

The following commercial enzyme-linked immunosorbent assay (ELISA) kits were used unless otherwise noted: pNF-H (BioVendor™), NSE (BioVendor™), GFAP (BioVendor™) and Tau/pTau (Meso Scale Siscovery™, MSD). Blinded blood and brain tissue sample analysis was conducted according to the manufacturer's instructions except as noted. All serum and brain tissues were analyzed using commercial kits except the GFAP levels in brain cortex and hippocampus were measured using MSD homebrew kits. For this biomarker, the operations were performed according to the manual provided by the manufacturer. Before running the ELISA tests, SULFO-Tag NNS was conjugated to mouse anti-GFAP cocktail (BD Pharmingen™) as a detector using reagents provided with the commercial homebrew kits. Briefly, as a capture antibody 25 μL of 0.5 μg/mL anti-GFAP monoclonal antibody cocktail (BD Pharmingen™) in phosphate-buffered saline (PBS) was coated in homebrew plates (MSD™) at 4° C. overnight. The next day, plates was blocked with TBST Start-Block™ buffer (Fisher™) followed by adding 25 μL of calibrator or sample. Recombinant human GFAP protein (Dx-SYS™) was used as a calibrator after serial dilution. Samples were diluted in TBST Start-Blocking™ buffer, if needed. After incubation at 4° C. overnight with shaking, plates were incubated with pre-prepared SULFO-Tag™ detector and read in a MSD™ microplate reader.

H. Histopathological Assessments

After behavioral testing, mice were anesthetized and perfused with 10% phosphate-buffered formalin. Brains were processed for frozen sectioning. Coronal slices were stained with hematoxylin and eosin for measurement of the lesion volume (all sites). Lesion volume (mm³) was determined by calculating the area of the lesion (mm²) and then by multiplying the sum of the lesion areas obtained from each section by the distance between sections (1 mm). Ipsilateral and contralateral hemispheric tissue volume was quantitated using the same approach. Both lesion volume and tissue volume loss were expressed as a percent of the contralateral (non-injured) hemisphere. A cohort of six mice for each time point and each group was analyzed. For unbiased evaluation of histopathology, images of the slides were scanned using an Aperio™ slide scanner (Leica Microsystems™ Inc.). The computed threshold was determined using Spectrum™ software. To ensure objective quantification, the same threshold was applied to all brain sections for each region of interest.

I. Elevated Plus Maze (EPM) Test of Anxiety

The elevated plus maze consists of two open arms and two closed arms. Anxious rodents avoid the open arms of the plus maze so that decreased time spent in and decreased entries into the open arms is a model system that reflects an enhanced level of anxiety. Mice were placed individually in the center of the maze (each arm was 33 cm long and 5 cm wide with 25 cm high walls on closed arms) and allowed free access for 5 minutes. Animals spent time either in a closed, safe, area (closed arms), in an open area (open arms) or in the middle, intermediate zone. Each session was videotaped with computer-based video tracking system (EthoVision™ XT 7.0, Noldus Information Technology™ Inc,) for later analysis by an observer blind to the experimental treatment. The apparatus was wiped with 70% ethanol and air-dried between mice. Recorded moving distance and the time spent in the open arms of the maze was analyzed with Student's t-test.

J. Morris Water Maze (MWM)

The MWM maze test was used as previously reported (see Yang et al., (24). Briefly, a water-filled pool was divided into four quadrants, each with a platform position equidistant from the center to the wall. During cue training that was used to assess the visual acuity and motor ability of the mice to escape the water to the platform independent of their spatial learning ability, the pool was filled to 1 cm below a visible plastic platform. During the spatial reference memory assessment (hidden platform training), the platform (12 cm diameter) was located in the southwest quadrant of the maze and submerged 1 cm below the surface of the water. During cue training, the platform and start positions were varied on each trial. Mice were given 6 trials at intervals of 10 minutes for two consecutive days. Beginning on the day after cue training was completed, mice received 5 consecutive days of hidden platform training (4 trials/day) to a hidden platform to assess spatial reference memory. The animals were allowed to search for the hidden platform for a period of 60 seconds, and the distance traveled to reach the platform was recorded. If an animal failed to find the hidden platform on any given trial, it was led there by the experimenter. As in cue training, mice were given a 10-minute inter-trial rest interval between trials for both training and probe trials. The start position for each trial (north, south, east, and west) varied on each trial. In the last day animals were tested in a probe trial in which the platform was removed from the pool and allowed to search for a period of 30 seconds. Swimming time in the target quadrant, where the platform previously had been placed was recorded. Each mouse's swimming episode was tracked and analyzed using a computer-based video tracking system (EthoVision™ XT 7.0, Noldus Information Technology™ Inc.).

K. Statistical Analysis

All statistical analyses were performed using GraphPad Prism™ (Version 5.0) software. Data were expressed as mean±SEM or median (interquartile range), as appropriate. The Wilcoxon matched pairs test or One-Way ANOVA was used, followed by post hoc analysis using Dunnett's test. All statistical tests were two-tailed and a p value <0.05 was considered significant.

### Example 2. Titers of Anti-GFAP Antibody Increase after Initial Immunization

Three sets of mice, corresponding to different time courses post-injury, each set including 15-20 mice, were studied (10 days post-injury (set 1), 20 days post injury (set 2) and 50 days post injury (set 3)). All mice received a first GFAP protein immunization (25 μg intact GFAP) at 7 weeks old and were boosted with the same dosage every two weeks for a total of three doses. Serum titers were measured by manifold immunoblotting in order to perform TBI surgery (CCI) at a time when the mice are expressing high anti-GFAP titers. See FIG. 1 for a flowchart showing the protocol.

Titers were measured by manifold immunoblotting. Mouse serum recognized a cluster of protein bands with molecular weights between 38 and 50 kDa (see FIG. 2A). These bands were identified as GFAP and its breakdown products (GBDP). The density of these bands indicated that only few mice showed increased anti-GFAP IgG at 20 days after the initial injection. Anti-GFAP IgG increased obviously at 30 day after the injection the increase lasted at least 50 days after three doses of immunization. See FIG. 2B. Anti-GFAP IgM was significantly elevated at Day 20, but to a lesser magnitude compared to anti-GFAP IgG at Day 30 and Day 50 (FIG. 2B). Thus, based on these results, the mouse CCI surgery was performed at post-immunization Day 30. See FIG. 1.

FIG. 2 is a temporal profile of anti-GFAP IgM and IgG titers as determined by manifold immunoblotting in mice after GFAP immunization. FIG. 2A is a set of representative images from the manifold immunoblot results. The 38-52 KD multiple bands are GFAP and GBDPs. Statistical analysis of the western blot results showed that anti-GFAP IgG increased after 30 days of immunization while no change of anti-GFAP IgM titer was observed. ** indicates p<0.01 compared to Day 0; *** indicates p<0.001 compared to Day 0; # indicates, for IgM, p<0.05 compared to Day 0.

Whether brain injury affects anti-GFAP antibody titers was tested next. Immunoblotting and ELISA were performed on serum samples collected from mice at different time points after CCI, and from control mice. Both mice receiving CCI and not receiving CCI had a long-lasting increase in anti-GFAP IgG after immunization until at least 50 days after injury even without additional injections. FIG. 3 presents anti-GFAP IgM, IgG titers in mice with or without CCI. Manifold immunoblotting (FIG. 3A) and ELISA (FIG. 3B) both showed increased anti-GFAP IgG or IgM compared to preimmunization. *** indicates p<00.1, ** indicates p<0.1, * indicates p<0.05. After CCI surgery, only anti-GFAP at D50 showed an increase compared to pre-CCI surgery (##p<0.01). CCI did not affect anti-GFAP IgM or IgG titers and the results were confirmed in mice without immunization (FIG. 3C). No change of GFAP titers was found in non-immunized mice with between pre- and post-CCI surgery. There was no difference between CCI mice and no CCI mice in terms of immune response (FIG. 3A and FIG. 3B). See Table 1 and Table 2 for statistical information for these data. One way ANOVA Dunett test and Wilcoxon matched pairs tests were used.

TABLE 1
Statistical Significance of Data, FIG. 3A.

| antibody | group | pre-CCI | 20d post-CCI | 50d post-CCI |
|---|---|---|---|---|
| GFAP-IgG | imm + CCI | * | ** | *** |
| | imm | * | *** | *** |
| GFAP-IgM | imm + CCI | *** | *** | ** |
| | imm | * | | |

compared to pre-immunization, *p <0.05, **p <0.01, ***p <0.001

TABLE 2
Statistical Significance of Data, FIG. 3B.

| antibody | group | pre-CCI | 20d post-CCI | 50d post-CCI |
|---|---|---|---|---|
| GFAP-IgG | imm + CCI | *** | *** | *** |
| | imm | *** | *** | *** |
| GFAP-IgM | imm + CCI | * | * | * |
| | imm | * | ** | ** |

compared to pre-immunization, *p <0.05, **p <0.01, ***p <0.001

Mice receiving no immunization also were tested. In this group, mice without CCI were compared to those with CCI, and were not different in either anti-GFAP IgG or IgM titers. This indicates that brain injury did not affect the immunization or trigger additional autoantibody response against GFAP in this model. See FIG. 3C. The IgG or IgM titers in unimmunized mice were less than 10 ng/mL, which is consistent with the pre-immunization titers shown in FIG. 3A and FIG. 3B. In addition, there were no differences in anti-GFAP IgG or anti-GFAP IgM titers between male and female mice.

### Example 3. Pre-Injury Immunization with GFAP Suppresses Astrocytes Activation Induced by TBI

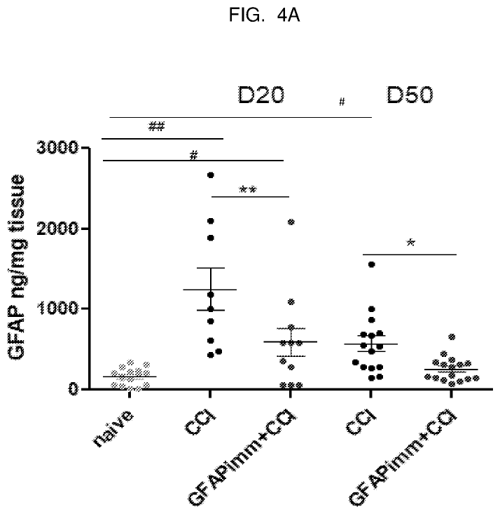

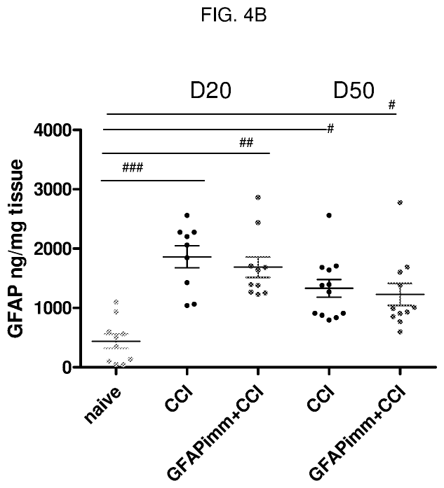

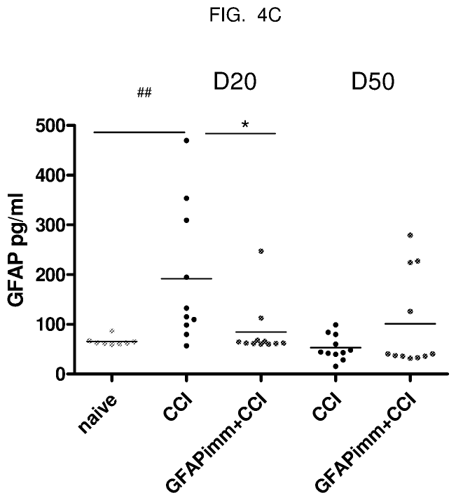

In the brain, astrocyte activation is a direct response to physical trauma to the brain. Astrocytes transiently becoming hypertrophic and express high levels of intermediate filament proteins such as GFAP. To measure the GFAP levels, MSD™ homebrew ELISA kits were used for tissue analysis and GFAP commercial kits were used for serum analysis. FIG. 4 shows that pre-immunization with GFAP suppressed astrocyte activation induced by TBI. GFAP expression in ipsilateral cortex (FIG. 4A) and ipsilateral hippocampus (FIG. 4B) as well as GFAP levels in serum (FIG. 4C) were monitored. Compared to the naïve group, # indicates p<0.05, ## indicates p<0.01, ### indicates p<0.001. Compared to the CCI group, * indicates p<0.05, ** indicates p<0.01.

GFAP is activated after brain injury via increased GFAP expression in the CCI mouse ipsilateral cortex at 20 days (median 1250.0±263.5 ng/mg protein) and 50 days (576.6±96.2 ng/mg) after CCI when compared to that of naïve mice (171.8±27.9 ng/mg). Pre-injury GFAP immunization with GFAP significantly reduced GFAP levels at both time points (Day 20: 595.4±178.5 vs 1250.0±263.5 ng/mg; Day 50: 261.8±39.38 vs 1250±263.5 ng/mg). FIG. 4A.

GFAP elevations also were found in ipsilateral hippocampus at 20 days (1863±186 ng/mg) and 50 days (1332±150 ng/mg) after CCI over that of naïve mice (439.8±117.7 ng/mg). See FIG. 4B. GFAP immunization, however, did not have any effects on hippocampal GFAP levels. Since the release of GFAP into serum is considered a biomarker of TBI, the levels of serum GFAP also were monitored. Serum GFAP levels increased at Day 20, then declined. At Day 20 after CCI, there was an increase of serum GFAP levels (192±43.79 pg/mL) when compared to naïve mice (65.66±3.122 pg/mL). At 20 days post-GFAP immunization, there was a significant attenuation of serum GFAP (84.19±16.93 pg/mL) (FIG. 4C). There was no difference in the GFAP levels in the CCI mice serum at 50 days post injury (with or without GFAP immunization) when compared to that in the naïve group (FIG. 4C).

### Example 4. Pre-Injury Immunization with GFAP Attenuates the Changes of pNF-H Levels after Brain Injury

Neurofilaments are exclusively found in the axons of neurons and mainly involved in maintaining neuronal shape and size and conduction of nerve impulses along the axons. Decreases in levels of neurofilament proteins in brain tissue reflect axonal degeneration. The levels of pNF-H in both brain tissues (ipsilateral cortex and hippocampus) and serum were measured. FIG. 5 shows data on pNF-H expression in ipsilateral cortex (FIG. 5A) and ipsilateral hippocampus (FIG. 5B) as well as GFAP levels in serum (FIG. 5C). Compared to the naïve group, ## indicates p<0.01; compared to the CCI group, ** indicates p<0.01.

In cortex, there was no significant change in pNF-H levels after CCI with or without immunization, when compared to naïve mice. See FIG. 5A. In contrast, there was a robust reduction in pNF-H levels in ipsilateral cortex and hippocampal pNF-H levels at D20 post injury, compared to that in naïve mice, while GFAP immunization significantly attenuated the pNF-H loss. See FIG. 5B. By Day 50, hippocampal pNF-H levels are similar to that in their naïve counterparts. In contrast, pNF-H significantly increased in mouse serum at 20 days and 50 days after injury, suggesting that due to the cell damage pNF-H released from the tissue into the biofluid. This is consistent with the reduced pNF-H found in hippocampus. FIG. 5C. The elevated pNF-H at Day 50 after CCI suggests continuing and sustained axonal degeneration into the chronic phase. Pre-treatment with GFAP immunization attenuated the declined pNF-H in serum at Day 50, suggesting neuroprotection or improved neuro recovery.

### Example 5. Pre-Injury GFAP Immunization Effects on NSE Levels in CCI Mice

FIG. 6 presents data showing that pre-injury GFAP immunization did not affect NSE levels in CCI mice (pNF-H in ipsilateral cortex (FIG. 6A), pNF-H in ipsilateral hippocampus (FIG. 6B) and pNF-H in serum (FIG. 6C). As shown in the figure, no changes of NSE levels in brain tissues (ipsilateral cortex or ipsilateral hippocampus) were observed when naive groups were compared to CCI groups at Day 20 and Day 50 (without or with GFAP immunization). A possible explanation is that NSE is an acute biomarker that indicates neuron death with a sharp increase 24 hours after injury followed by a decrease over time. Consistent with this theory, several mice did maintain high levels of serum NSE at Day 20 (see FIG. 6C), though this was not statistically significance.

### Example 6. Pre-Injury Immunization Protective Effects on Tauopathy-Linked Neurodegeneration

Tau and its hyperphosphorylated form (P-Tau) play a major role in neurodegenerative disease, mediating neural cell death. Increasing evidence show tauopathy could be a chronic manifestation of TBI such as chronic traumatic encephalopathy (CTE). Tau and P-Tau also are emerging as potential TBI biomarkers.

Tau and P-Tau levels were analyzed in CCI mice with or without GFAP immunization. MSD™ commercial kits were used to measure the concentrations of total Tau and P-Tau proteins. Neither total Tau nor P-Tau differed significantly in either ipsilateral cortex or ipsilateral hippocampus between these two groups at Day 20 post-injury. In contrast, at Day 50 post-injury, both total Tau and P-Tau protein levels were increased when compared to the Day 20 counterpart. Also, GFAP immunization resulted in significantly decreased total Tau and P-Tau levels in the ipsilateral cortex (FIG. 7). No changes of total Tau levels in hippocampus was observed among groups, while there was a trend of decrease in P-Tau at Day 50 with GFAP immunization (p=0.13). Since P-tau is postulated to be related to tauopathy formation, P-Tau/T-Tau ratio is also considered to be an important index.

Data were analyzed using a Wilcoxon matched pair test compared to CCI. Statistical analysis on P-Tau/T-Tau ratio showed that GFAP immunization significantly decreased this ratio in both cortex and hippocampus, indicating GFAP immunization could prevent tauopathy formation (FIG. 7). Total Tau levels in cortex and P-Tau levels in hippocampus were even higher at Day 50 compared to that at Day 20. Tau hyperphosphorylation in cortex also exhibited a strong increase trend (p=0.068) at Day 50. These findings suggested a long-lasting tauopathy after TBI and that pre-injury immunization with GFAP has protective effects on tauopathy-linked neurodegeneration. In FIG. 7, # indicates p<0.05 compared to Day 20 post injury; * indicates p<0.05 compared to CCI mice; ** indicates p<0.01 compared to CCI mice; and *** indicates p<0.001 compared to CCI mice.

### Example 7. Pre-Immunization with GFAP has No Benefit on Histopathological Outcomes

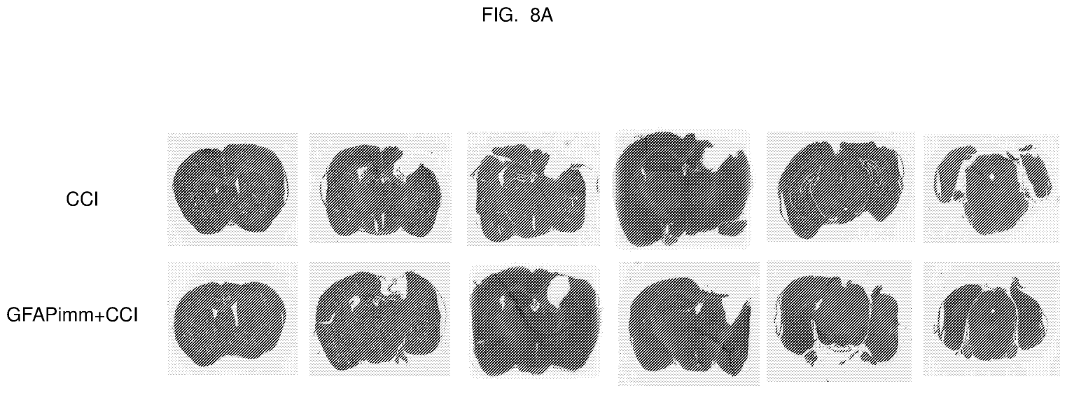

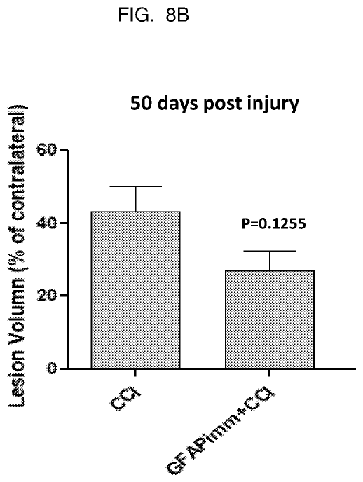

Comparisons of gross histopathological measurements are shown in FIG. 8. This includes representative sections from each group for general comparison (FIG. 8A) and the pooled analyses results for lesion volume (FIG. 8B). These detailed serial section images allow visualization of the anatomic location of the damage in each group. Results of the Wilcoxon matched-pair test for mean percent change in the ipsilateral cerebral cortex relative to the contralateral (uninjured) side showed a trend of benefit in lesion volume in the GFAP immunization group, but this effect was not significant (p=0.1255). See FIG. 8, which shows the extent and placement of the lesion (FIG. 8A) and the lesion volume (FIG. 8B). The data represent group means±standard error of the mean.

### Example 8. GFAP Immunization Alleviate Post-Injury Anxiety and Improves Cognitive Functions

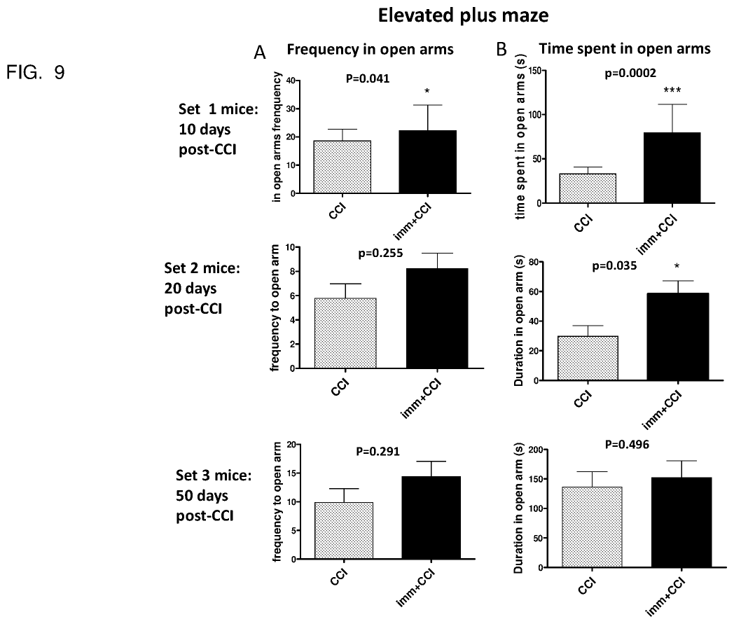

Post-TBI anxiety-like behavior was examined using the elevated plus maze (EMP) test, which is followed by the cognitive and memory (Morris water maze (MWM)) test on three individual sets of mice. Each mouse only experienced once behavioral test. Three time courses after injury were used: 10 days, 20 days and 50 days post-CCI surgeries. See FIG. 9 for the results. FIG. 9A shows the frequency in open arms; FIG. 9B shows the time spent in open arms. In the EMP test, at 10 days post-CCI, the GFAP pre-immunization group had a significantly higher frequency of entering the open arms and spent more time in the open arms, indicating this group mice presented less anxious behavior. At 20 days, mice undergoing GFAP immunization still had more duration in the open arms. However this benefit did not last to 50 days.

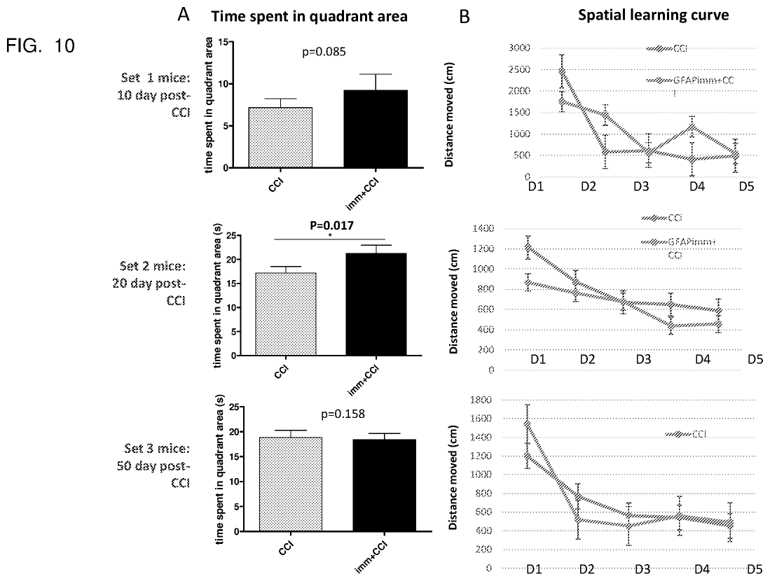

Following EMP tests, 5 days of MWM tests were performed to test the behavioral function in mice, evaluated at 10, 20, and 50 days post CCI. The CCI model was relatively modest in terms of inducing a deficit in latency to find the hidden platform in the MWM test. See FIG. 10 for the results. FIG. 10A shows the time spent in the correct quadrant area, indicating the memory function; FIG. 10B shows the spatial learning curve, related to the spatial leaning function. At 10 days, mice which underwent GFAP immunization showed a trend of increased memory function (p=0.085) by spending more time in the quadrant area. The only significant effect of GFAP immunization on MWM test outcomes was an improvement in memory function at 20 days post injury. However, there was no such effect at 50 days (see FIG. 10A). No effects were observed on spatial learning at these three time points (see FIG. 10B). Thus, pre-immunization with GFAP protein improved cognitive deficits, but did not improve spatial learning. In FIG. 10, * indicates p<0.05 compared to the CCI group.

### Example 9. Intact Full Length and Calpain-Truncated GFAP (GBDP) have Neurotoxic Effects on Rat Cerebrocortical Culture (CTX)

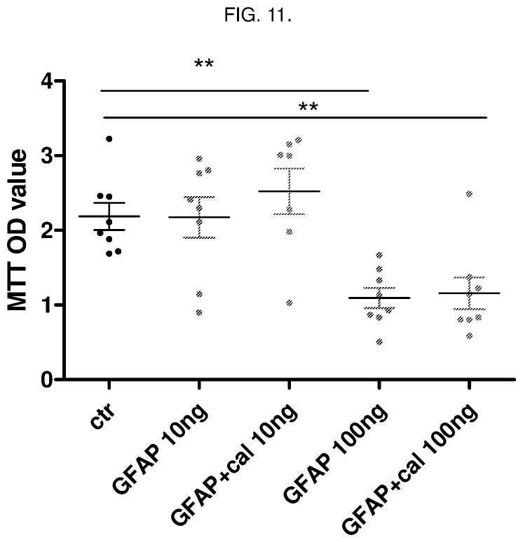

CTX cultures in 96-well culture plates were treated with 10 ng or 100 ng full length and calpain-truncated GFAP (GBDP) protein in 100 μL media for 16 hours. Cell viability was assessed by the mitochondrial uptake (reduction of dye 3-(4,5-dimethylthiazol-2-yl)-2,5-diphenyltetrazolium (MTT) by functional cellular mitochondria (shown are optional density (ODD values at 490 nm)). Data presented in FIG. 11 show that full length and calpain-truncated GFAP (GBDP) at 100 ng/100 μL induced neurotoxicity in these CTX neuronal cultures, as shown by robust and significant reduction of mitochondrial function measured by MTT dye reduction.

Supplemental Methods Related to Examples 10-14

Methods:

Post-Traumatic Brain Injury GFAP Antibody Treatment in Mice:

Mouse strain: C57BL/6 mice are used. For Post-traumatic brain injury antibody treatment in mice Mouse MAb (BD Pharmingen—Purified Mouse monoclonal Anti-GFAP antibody cocktail (clones 1B4, 4A11, 2E1) Catalog No. 556330; concentration 0.5 mg/ml) was used. The study has three arms. Arm 1—controlled cortical impact (CCI, as a form of experimental TBI+saline (N=12), Arm 2—CCI+GFAP MAb (N=12). On day 1, immediately after CCI, immediate bolus dose of Purified GFAP Mab (mouse monoclonal antibody) in 0.9% saline via orbital vein (facial) at 20 ug/C57BL/6 mouse (approx. 25 g by weight) was given, followed by same dose at day 3, 7, 14, 21 and 28.

As an alternative follow-up MAb administration, following the initial bolus dose of anti-GFAP Mab via the orbital vein, an ALZET osmotic pump is implanted subcutaneously following the implant protocol of the ALZET osmotic pump (cat #1004). Briefly, a mid-scapular incision is made with 1.0-1.5 cm longer than the pump length. Use a hemostat into the incision to create a pocket. a filled pump is placed into the pocket, and then the incision is closed with sutures. GFAP Mab used (20 μg) is diluted in total 100 μL with 0.9% saline and pumping rates is 0.11 μL/hr.

For assessment, Day 3, Day 7 (200 μL) Serum samples are obtained as well as terminal (Day 30) serum samples (1 mL) after neurobehavioral assessment (which include elevated plus maze/EPM for anxiety like behavior assessment and Y-Maze and Morris water maze (MWM)—both as cognitive/memory function assessments. Brain tissue are pulverized and lysed with Triton-X-100 (1%) lysis buffer containing 50 mM Tris-HCl, 5 mM EDTA, 1 mM dithiothreitol and protease and phosphatase inhibitor cocktail (EMD Bioscience). Brain tissue (ipsilateral, contralateral cortex or hippocampus are analyzed for brain biomarker protein levels using enzyme linked immunosorbent assay (ELISA) or denaturing-gel electrophoresis following with electrotransfer and immunoblotting with antibody against neurobiomarkers—and enzyme (alkaline phosphatase)-substrate based colorimetric development.

### Example 10. Anti-GFAP MAb Immunization Decreases Anxiety Like Behavior in Mice after Controlled Cortical Impact (CCI)

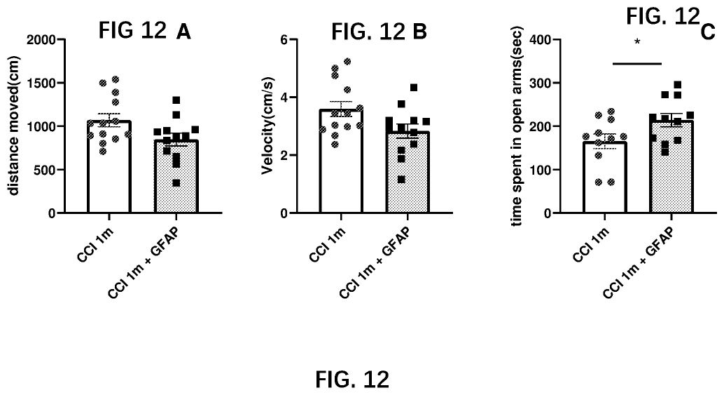

At 1 mo. from mice subjected to controlled cortical impact (CCI)—a form of TBI, without or with GFAP MAb therapy, mice were subjected to EPM test. As shown in FIG. 12, the distance traveled (FIG. 12A) and velocity of mouse movement (FIG. 12B) for both CCI 1m and CCI 1 m+anti-GFAP Mab group are the same. On the other hand, mice in the CCI+GFAP MAb group spent more time in the open arms, which demonstrates that anti-GFAP MAb therapy reduced anxiety behavior.

### Example 11. Anti-GFAP MAb Immunization Increases Cognitive Function and Memory after CCI

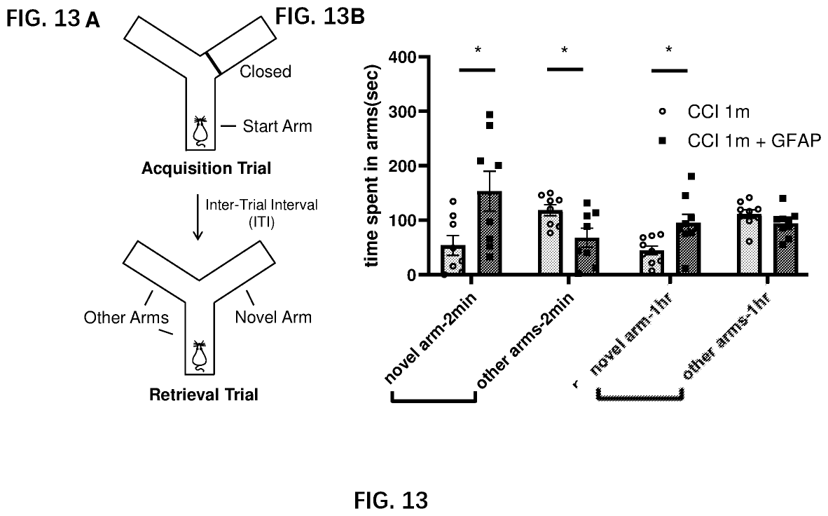

At 30 day (1 mo.) from mice being subjected to CCI, without or with GFAP MAb therapy, the mice were tested using a Y-maze. FIG. 13A shows the Y-maze set-up. Mice were first trained in the acquisition trail with one arm closed. Then after 2 min and also 1 hour inter-trial interval (ITI), the mice are subjected to the retrieval trial (twice). For the retrieval trials conducted at both 2 min ITI and at 1 h ITI, the CCI 1 mo.+GFAP Mab group spent more time than the CCI 1 mo. group in the novel arm. In addition, at 2 min ITI, the GFAP group also spent less time in the other two arms (* p<0.05); n=7-8 (See FIG. 13B).

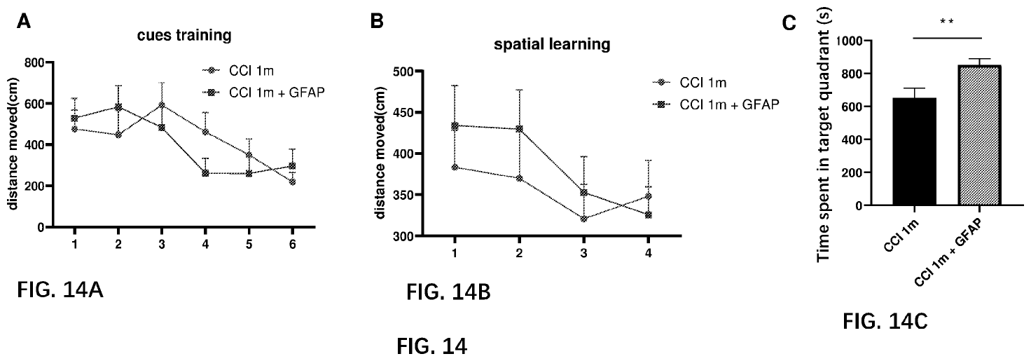

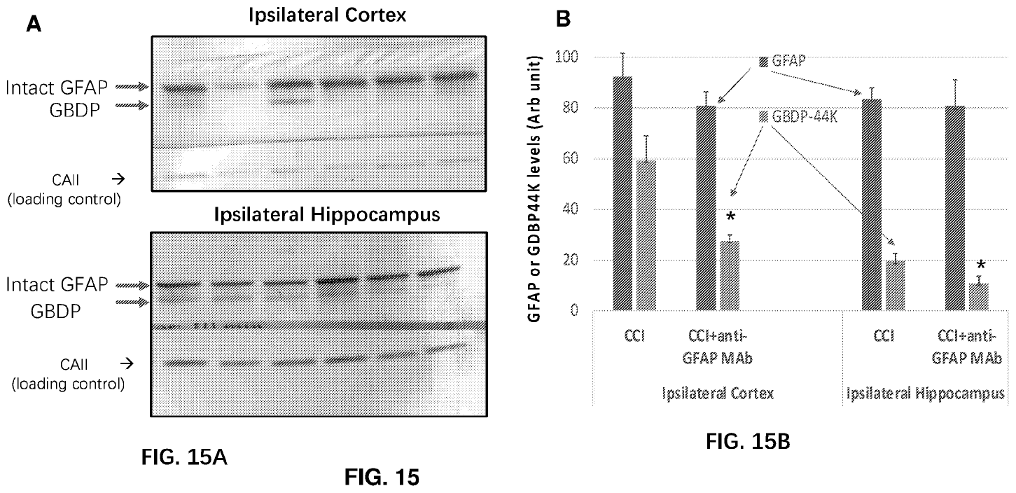

Turning to FIG. 15, the CCI 1 mo and CCI 1 mo+GFAP MAb were tested using the Morris Water Maze. At 24-30 day (1 mo.) from mice subjected to CCI, without or with GFAP MAb therapy, mice are subjected to MWM cue training (FIG. 14AA) and spatial learning (FIG. 14B) and then subjected to probe trial (FIG. 14C). At training/learning stage, both CCI 1 mo. and CCI 1 mo.+GFAP MAb groups exhibited the same pattern in distance moved during both cues training stage and spatial learning stage. At probe trial stage, the CCI 1 mo.+GFAP Mab group spent more time than the CCI 1 mo. group in the target quadrant area (** p<0.01); n=12.

### Example 12. Anti-GFAP Antibody Immunization Decreases GBDP Levels in Brain Following CCI

FIG. 15 shows that post-injury immunization therapy with mouse anti-GFAP antibody suppressed GBDP levels (panel A shows immunoblotting of ipsilateral cortex IC) and hippocampus (IH), respectively. Immunoblots probed with anti-GFAP antibodies show the relative levels of GBDP (mainly 40 kDa) in addition to intact GFAP (50 kDa) N=3. Panel B is densitometric quantification of both intact GFAP and GFAP breakdown product ( ) bands (mean+SEM). The intact GFAP levels are the same for both CCI and CCI+GFAP MAb groups. However, the levels of GBDP in both ipsilateral cortex and hippocampus were significantly attenuated in the CCI+GFAP MAb group. Since it was conceptualized that GBDP is first produced by TBI (CCI) induced calpain protease activation in injured astrocytes, then GBDP is released into extracellular fluid and might have neurotoxic effects. This data shows that systemic GFAP Mab treatment in fact has the capacity to fulfil target engagement by reaching this extracellular pool of GBDP in the brain and subsequently reducing its load presumably by IgG mediated phagocytosis/clearance by microglia and macrophage.

### Example 13. Anti-GFAP MAb Immunization Attenuates P-Tau-/Total Tau Ratio in Brain Tissue

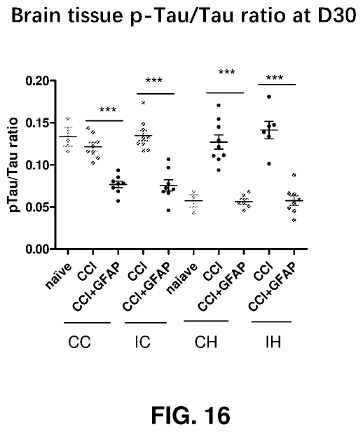

FIG. 16 shows post-injury immunization therapy with mouse anti-GFAP MAb antibody attenuated P-Tau/Total Tau ratio in brain tissue naïve mouse group has n=4 (for comparison), CCI and CCI+GFAP MAb groups have n=8. At 1 mo. (30 days) post-injury, brain tissue from different regions were used to prepare brain lysate that are equalized by protein assay to 1 mg/mL: IC, IH are ipsilateral cortex and hippocampus, and CC, CH are contralateral cortex and hippocampus, respectively. (* p<0.05, ** p<0.01, * p<0.001), Both Total Tau and P-Tau (Thr-231) were assayed with the mesoscale Discovery (MSD) duplex kit. Data shown are mean+/−SEM. In all tissue samples, the ratio of P-Tau/T-Tau was reduced about 2-fold. Since P-Tau is associated with post-TBI neurodegeneration and tauopathy. These effects of GFAP MAb treatment are interpreted as neuroprotective and anti-neurodegeneration.

### Example 14. Anti-GFAP MAb Immunization Reduces Tau Released into Circulation

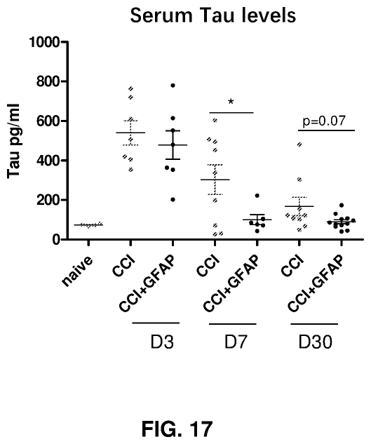

FIG. 17 shows post-injury immunization therapy with anti-GFAP MAb antibody reduced Tau released into circulation (serum fraction). naïve mouse group has n=4 (for comparison), CCI and CCI+GFAP MAb groups have n=7-10. At day 3, day 7 and D30 (1 mo.) post-injury, blood samples were collected and processed to serum fraction. Tau was measured using high sensitivity Quanterix mouse tau kit (it is noted that P-Tau mouse tau kit was not available for use at the time of this study—thus P-Tau in serum samples was not measured). Data shown are mean+/−SEM. There were strong elevations of Tau at all three time points compared to naïve, especially in Day 3 and 7. By D7 of GFAP MAb treatment, the levels of released Tau were significantly attenuated (* p<0.05). Since Tau release into blood is associated with post-TBI neurodegeneration and tauopathy, these effects of GFAP MAb treatment are interpreted as neuroprotective and anti-neurodegeneration.

Taken together, Examples 10-14 shows that post-TBI immunotherapy treatment with anti-GFAP monoclonal antibody for about 28 days improve neurobehavioral functional recovery in mice. In addition, brain tissue and blood-based neuroinjury biomarkers are attenuated by anti-GFAP monoclonal antibody treatment.

### REFERENCES

References listed below and throughout the specification are hereby incorporated by reference in their entirety.
1. Ahmad K, Baig M H, Mushtaq G, Kamal M A, Greig N H, Choi I. Commonalities in Biological Pathways, Genetics, and Cellular Mechanism between Alzheimer Disease and Other Neurodegenerative Diseases: An In Silico-Updated Overview. Curr Alzheimer Res. 2017 Oct. 6; 14(11):1-16.
2. An C, Jiang X, Pu H, Hong D, Zhang W, Hu X, Gao Y (2016) Severity-Dependent Long-Term Spatial Learning-Memory Impairment in a Mouse Model of Traumatic Brain Injury. Translational Stroke 7:512-520.
3. Bae J R, Kim S H. Synapses in neurodegenerative diseases. BMB Rep. Korean Society for Biochemistry and Molecular Biology; 2017 May; 50(5):237-46.
4. Baldwin G, Breiding M, Sleet D (2016) Using the public health model to address unintentional injuries and TBI: A perspective from the Centers for Disease Control and Prevention (CDC). NeuroRehabilitation 39:345-349.
5. Bell K, Wilding C, Funke S, Perumal N, Beck S, Wolters D, Holz-Müller J, Pfeiffer N, Grus F H (2016) Neuroprotective effects of antibodies on retinal ganglion cells in an adolescent retina organ culture. Journal of Neurochem. 139:256-269.
6. Bittar A, Sengupta U, Kayed R (2018) Prospects for strain-specific immunotherapy in Alzheimer's disease and tauopathies. NPJ Vaccines 3:9.
7. Biundo F, Del Prete D, Zhang H, Arancio O, D'Adamio L (2018) A role for tau in learning, memory and synaptic plasticity. Sci Rep 8:3184.
8. Boutajangout A, Ingadottir J, Davies P, Sigurdsson E M (2011) Passive immunization targeting pathological phospho-tau protein in a mouse model reduces functional decline and clears tau aggregates from the brain. Journal of Neurochem. 118:658-667.
9. Boutajangout A, Quartermain D, Sigurdsson E M (2010) Immunotherapy targeting pathological tau prevents cognitive decline in a new tangle mouse model. The Journal of Neurosci. 30:16559-16566.
10. Boutté A M, Deng-Bryant Y, Johnson D, Tortella F C, Dave J R, Shear D A, Schmid K E (2015) Serum Glial Fibrillary Acidic Protein Predicts Tissue Glial Fibrillary Acidic Protein Break-Down Products and Therapeutic Efficacy after Penetrating Ballistic-Like Brain Injury. J Neurotrauma 33:147-156.
11. Bramlett H M, Dietrich W D, Dixon C E, Shear D A, Schmid K E, Mondello S, Wang K K W, Hayes R L, Povlishock J T, Tortella F C, Kochanek P M (2015) Erythropoietin Treatment in Traumatic Brain Injury: Operation Brain Trauma Therapy. J Neurotrauma 33:538-552.
12. Browning M, Shear D A, Bramlett H M, Dixon C E, Mondello S, Schmid K E, Poloyac S M, Dietrich W D, Hayes R L, Wang K K W, Povlishock J T, Tortella F C, Kochanek P M (2015) Levetiracetam Treatment in Traumatic Brain Injury: Operation Brain Trauma Therapy. J Neurotrauma 33:581-594.
13. Castillo-Carranza D L, Guerrero-Muñoz M J, Sengupta U, Hernandez C, Barrett A D T, Dineley K, Kayed R (2015) Tau immunotherapy modulates both pathological tau and upstream amyloid pathology in an Alzheimer's disease mouse model. The Journal of . . . 35:4857-4868.
14. Cox A L, Coles A J, Nortje J, Bradley P G, Chatfield D A, Thompson S J, Menon D K (2006) An investigation of auto-reactivity after head injury. J Neuroimmunol 174:180-186.
15. Crane P K, Gibbons L E, Dams-O'Connor K, Trittschuh E, Leverenz J B, Keene C D, et al. Association of Traumatic Brain Injury With Late-Life Neurodegenerative Conditions and Neuropathologic Findings. JAMA Neurol. 2016 Sep. 1; 73(9):1062-8.
16. Dai C-L, Chen X, Kazim S F, Liu F, Gong C-X, Grundke-Iqbal I, Iqbal K (2014) Passive immunization targeting the N-terminal projection domain of tau decreases tau pathology and improves cognition in a transgenic mouse model of Alzheimer disease and tauopathies. J Neural Transm (Vienna) 122:607-617.
17. Dixon C E, Bramlett H M, Dietrich W D, Shear D A, Yan H Q, Deng-Bryant Y, Mondello S, Wang K K W, Hayes R L, Empey P E, Povlishock J T, Tortella F C, Kochanek P M (2015) Cyclosporine Treatment in Traumatic Brain Injury: Operation Brain Trauma Therapy. J Neurotrauma 33:553-566.
18. Dossi E, Vasile F, Rouach N. Human astrocytes in the diseased brain. Brain Research Bulletin. Elsevier Inc; 2018 Jan. 1; 136:139-56.
19. Eisenried A, Meidahl A C N, Klukinov M, Tzabazis A Z, Sabbadini R A, Clark J D, Yeomans D C (2017) Nervous system delivery of antilysophosphatidic acid antibody by nasal application attenuates mechanical allodynia after traumatic brain injury in rats. Pain 158:2181-2188.
20. Eng L F, Ghirnikar R S, Lee Y L (2000) Glial fibrillary acidic protein: GFAP-thirty-one years (1969-2000). Neurochem Res 25:1439-1451.
21. Gardner R C, Byers A L, Barnes D E, Li Y, Boscardin J, Yaffe K. Mild TBI and risk of Parkinson disease. Neurology. 2018 May 14; 90(20):e1771-9.
22. Garwood C J, Ratcliffe L E, Simpson J E, Heath P R, Ince P G, Wharton S B. Review: Astrocytes in Alzheimer's disease and other age-associated dementias: a supporting player with a central role. Neuropathol Appl Neurobiol. 2017 May 22; 43(4):281-98.
23. Goryunova A V, Bazarnaya N A, Sorokina E G (2007) Glutamate receptor autoantibody concentrations in children with chronic post-traumatic headache. Neuroscience and Behav. Physiol. 37:761.
24. Hayashi N, Miyata S, Kariya Y, Takano R, Hara S, Kamei K. Attenuation of glial scar formation in the injured rat brain by heparin oligosaccharides. Neurosci Res. 2004 May; 49(1):19-27.
25. Hu R, Zhou J, Luo C, Lin J, Wang X, Li X, Bian X, Li Y, Wan Q, Yu Y, Feng H (2010) Glial scar and neuroregeneration: histological, functional, and magnetic resonance imaging analysis in chronic spinal cord injury. J Neurosurg Spine 13:169-180.
26. Jassam Y N, Izzy S, Whalen M, McGavern D B, Khoury El J. Neuroimmunology of Traumatic Brain Injury: Time for a Paradigm Shift. Neuron. Elsevier Inc; 2017 Sep. 13; 95(6):1246-65.
27. Jordan B D. The clinical spectrum of sport-related traumatic brain injury. Nat Clin Pract Neurol. 2013 Mar. 12.
28. Kou Z, Vandevord P J. Traumatic white matter injury and glial activation: From basic science to clinics. Matute C, Stys P K, editors. Glia. 2014 May 7; 62(11):1831-55.
29. Lacosta A-M et al. (2018) Safety, tolerability and immunogenicity of an active anti-Aβ40 vaccine (ABvac40) in patients with Alzheimer's disease: a randomised, double-blind, placebo-controlled, phase I trial. Alzheimers Res Ther 10:12.
30. Lucke-Wold B, Seidel K, Udo R, Omalu B, Ornstein M, Nolan R, Rosen C, Ross J (2017) Role of Tau Acetylation in Alzheimer's Disease and Chronic Traumatic Encephalopathy: The Way Forward for Successful Treatment. J Neurol Neurosurg 4.
31. Marchi N, Bazarian J J, Puvenna V, Janigro M (2013) Consequences of repeated blood-brain barrier disruption in football players. PloS one 8:e56805.
32. McKee A C, Daneshvar D H (2015) The neuropathology of traumatic brain injury. Handb Clin Neurol 127:45-66.
33. McKee A C, Stein T D, Kiernan P T, Alvarez V E (2015) The neuropathology of chronic traumatic encephalopathy. Brain Pathol 25:350-364.
34. McPherson R C, Anderton S M (2013) Adaptive immune responses in CNS autoimmune disease: mechanisms and therapeutic opportunities. J Neuroimmune Pharmacol 8:774.
35. Mielke M M, Hagen C E, Wennberg A M V, Airey D C, Savica R, Knopman D S, Machulda M M, Roberts R O, Jack C R, Petersen R C, Dage J L (2017) Association of Plasma Total Tau Level With Cognitive Decline and Risk of Mild Cognitive Impairment or Dementia in the Mayo Clinic Study on Aging. JAMA Neurol 74:1073-1080.
36. Mondello S, Kobeissy F, Vestri A, Hayes R L, Kochanek P M, Berger R P (2016) Serum Concentrations of Ubiquitin C-Terminal Hydrolase-L1 and Glial Fibrillary Acidic Protein after Pediatric Traumatic Brain Injury. Sci Rep 6:28203.
37. Mountney A, Bramlett H M, Dixon C E, Mondello S, Dietrich W D, Wang K K W, Caudle K, Empey P E, Poloyac S M, Hayes R L, Povlishock J T, Tortella F C, Kochanek P M, Shear D A (2016) Simvastatin Treatment in Traumatic Brain Injury: Operation Brain Trauma Therapy. J Neurotrauma 33:567-580.
38. Naegele M, Martin R (2014) The good and the bad of neuroinflammation in multiple sclerosis. Handb Clin Neurol 122:59-87.
39. Neselius S, Zetterberg H, Blennow K, Marcusson J, Brisby H (2013) Increased CSF levels of phosphorylated neurofilament heavy protein following bout in amateur boxers. PloS one 8:e81249.
40. Olivera A, Lejbman N, Jeromin A, French L M, Kim H-S, Cashion A, Mysliwiec V, Diaz-Arrastia R, Gill J (2015) Peripheral Total Tau in Military Personnel Who Sustain Traumatic Brain Injuries During Deployment. JAMA Neurol 72:1109-1116.
41. Ordóñez-Gutiérrez L, Posado-Fernàndez A, Ahmadvand D, Lettiero B, Wu L, Antón M, Flores O, Moghimi S M, Wandosell F (2016) ImmunoPEGliposome-mediated reduction of blood and brain amyloid levels in a mouse model of Alzheimer's disease is restricted to aged animals. Biomaterials 112:141-152.
42. Pang Z, Higuchi M, Koriyama H, Yoshida S, Kurinami H, Shimamura M, Takami Y, Rakugi H, Morishita R, Nakagami H (2016) Evaluating the potential of the GFAP-KLH immune-tolerizing vaccine for type 1 diabetes in mice. FEBS Lett 591:129-136.
43. Pekny M, Wilhelmsson U, Pekna M. Neuroscience Letters. Neurosci Lett. Elsevier Ireland Ltd; 2014 Apr. 17; 565:30-8.
44. Pekny M, Pekna M. Reactive gliosis in the pathogenesis of CNS diseases. BBA—Molecular Basis of Disease. Elsevier B.V; 2016 Mar. 1; 1862(3):483-91.
45. Pekny M, Wilhelmsson U, Tatlisumak T, Pekna M. Astrocyte activation and reactive gliosis—A new target in stroke? Neurosci Lett. Elsevier; 2018 Aug. 2; 1-0.
46. Pinelis V G, Sorokina E G, Semenova J B, Karaseva O V, Mescheryakov S V, Chernisheva T A, Arsenieva E N (2015) [Biomarkers in children with traumatic brain injury]. Zh. Nevrol. Psikhiatr. Im S S Korsakova 115:66-72.
47. Ramos-Cejudo J, Wisniewski T, Marmar C, Zetterberg H, Blennow K, de Leon M J, et al. Traumatic Brain Injury and Alzheimer's Disease: The Cerebrovascular Link. EBIOM. The Authors; 2018 Feb. 1; 28(C):21-30.
48. Rubenstein R et al. (2017) Comparing Plasma Phospho Tau, Total Tau, and Phospho Tau-Total Tau Ratio as Acute and Chronic Traumatic Brain Injury Biomarkers. JAMA Neurol 74:1063-1072.
49. Rudehill S, Muhallab S, Wennersten A, Gertten von C, Nimer Al F, Sandberg-Nordqvist A C, et al. Autoreactive antibodies against neurons and basal lamina found in serum following experimental brain contusion in rats. Acta Neurochir. 2005 Dec. 20; 148(2):199-205.
50. Sankaranarayanan S et al. (2015) Passive immunization with phospho-tau antibodies reduces tau pathology and functional deficits in two distinct mouse tauopathy models. PloS one 10:e0125614.
51. Santacruz K, Lewis J, Spires T, Paulson J, Kotilinek L, Ingelsson M, Guimaraes A, DeTure M, Ramsden M, McGowan E, Forster C, Yue M, Orne J, Janus C, Mariash A, Kuskowski M, Hyman B, Hutton M, Ashe K H (2005) Tau suppression in a neurodegenerative mouse model improves memory function. Science 309:476-481.
52. Sarkar S, Keswani T, Sengupta A, Mitra S, Bhattacharyya A (2017) Differential modulation of glial cell mediated neuroinflammation in Plasmodium berghei ANKA infection by TGF β and IL 6. Cytokine 99:249-259.
53. Schafer M K E, Tegeder I. NG2/CSPG4 and progranulin in the posttraumatic glial scar. Matrix Biol. 2017 Oct. 17.
54. Schwartz M (2017) Can immunotherapy treat neurodegeneration? Science 357:254-255.
55. Seo J-S et al. (2017) Transcriptome analyses of chronic traumatic encephalopathy show alterations in protein phosphatase expression associated with tauopathy. Exp Mol Med 49:e333.
56. Shaw G, Yang C, Ellis R, Anderson K, Mickle J P, Scheff S, Pike B, Anderson D K, Howland D R (2005) Hyperphosphorylated neurofilament N F-H is a serum biomarker of axonal injury. Biochem Biophys Res Commun 336:1268-1277.
57. Shear D A, Dixon C E, Bramlett H M, Mondello S, Dietrich W D, Deng-Bryant Y, Schmid K E, Wang K K W, Hayes R L, Povlishock J T, Kochanek P M, Tortella F C (2015) Nicotinamide treatment in traumatic brain injury: operation brain trauma therapy. J Neurotrauma 33:523-537.
58. Shibahashi K, Doi T, Tanaka S, Hoda H, Chikuda H, Sawada Y, Takasu Y, Chiba K, Nozaki T, Hamabe Y, Ogata T (2016) The Serum Phosphorylated Neurofilament Heavy Subunit as a Predictive Marker for Outcome in Adult Patients after Traumatic Brain Injury. J Neurotrauma 33:1826-1833.
59. Sofroniew M V. Molecular dissection of reactive astrogliosis and glial scar formation. Trends in neurosciences [Internet]. 2009 December; 32(12):638-47.
60. Sorokina E G, Semenova Z B, Granstrem O K (2009) [5100B protein and autoantibodies to S100B protein in diagnostics of brain damage in craniocerebral trauma in children]. Zh. Nevrol. Psikhiatr. Im S S Korsakova 110:30-35.
61. Vellas B, Black R, Thal L J, Fox N C, Daniels M, McLennan G, Tompkins C, Leibman C, Pomfret M, Grundman M (2009) Long-term follow-up of patients immunized with AN1792: reduced functional decline in antibody responders. Curr Alzheimer Res 6:144-151.
62. Verkhratsky A, Nedergaard M, Hertz L. Why are Astrocytes Important? Neurochem Res. 2014 Aug. 12; 40(2):389-401.
63. Volterra A, Meldolesi J (2005) Astrocytes, from brain glue to communication elements: the revolution continues. Nature Reviews Neuroscience 6:626-640.
64. Wang K, Yang Z, Yue J K, Zhang Z (2016) Plasma Anti-Glial Fibrillary Acidic Protein Autoantibody Levels during the Acute and Chronic Phases of Traumatic Brain Injury: A Transforming Research and Clinical . . . J Neurotrauma 33:1270.
65. Wang K K, Yang Z, Zhu T, Shi Y, Rubenstein R, Tyndall J A, Manley G T (2018) An update on diagnostic and prognostic biomarkers for traumatic brain injury. Expert Rev Mol Diagn 18:165-180.
66. Wilding C, Bell K, Funke S, Beck S, Pfeiffer N, Grus F H (2015) GFAP antibodies show protective effect on oxidatively stressed neuroretinal cells via interaction with ERP57. J Pharmacol Sci 127:298-304.
67. Wilson L, Stewart W, Dams-O'Connor K, Diaz-Arrastia R, Horton L, Menon D K, et al. The chronic and evolving neurological consequences of traumatic brain injury. The Lancet Neurology. 2017 October; 16(10):813-25.
68. Yanamandra K et al. (2017) Anti-tau antibody administration increases plasma tau in transgenic mice and patients with tauopathy. Sci Transl Med 9:eaa12029.
69. Yang Z, Lin F, Robertson C S, Wang K K W (2014) Dual vulnerability of TDP-43 to calpain and caspase-3 proteolysis after neurotoxic conditions and traumatic brain injury. J Cereb Blood Flow Metab 34:1444-1452.
70. Yang Z, Zhu T, Weissman A S, Jaalouk E, Rathore D S, Romo, P, Shhi Y, Wagner A K, Wang K K (2018) Autoimmunity and traumatic brain injury. Curr Phys Med Rehabil Rep 5:22-29.
71. Yang Z, Wang K (2015) Glial fibrillary acidic protein: from intermediate filament assembly and gliosis to neurobiomarker. Trends Neurosci. 38(6):364-374.
72. Yang Z, Wang P, Morgan D, Lin D, Pan J, Lin F, Strang K H, Selig T M, Perez P D, Febo M, Chang B, Rubenstein R, Wang K K W (2015) Temporal MRI characterization, neurobiochemical and neurobehavioral changes in a mouse repetitive concussive head injury model. Sci Rep 5:11178.
73. Zhang Z, Zoltewicz J S, Mondello S, Newsom K J (2014a) Human traumatic brain injury induces autoantibody response against glial fibrillary acidic protein and its breakdown products. PloS one 9:e92698.
74. Zhao Z, Loane D J, Murray M G, Stoica B A, Faden A I (2012) Comparing the predictive value of multiple cognitive, affective, and motor tasks after rodent traumatic brain injury. J Neurotrauma 29:2475-2489.
75. Zhu G, Chen X (2018) Aptamer-based targeted therapy. Adv Drug Deliv Rev 134:65-78.
76. Zurek J, Bartlová L, Fedora M (2011) Hyperphosphorylated neurofilament NF-H as a predictor of mortality after brain injury in children. Brain Injury 25:221-226.
77. International Patent Publication No. WO/2010/004432A1 to Lafaye et al.
78. Chinese Patent CN 20150490145 to Yang et al.
79. U.S. Pat. No. 8,372,808 to Messing et al.
80. United States Patent Application No. 2016-0303068 to Cross et al.
81. United States Patent Application No. 2018-003157 to Wang et al.
82. International Patent Publication No. WO/2011/160096.

What is claimed is:

1. A method of suppressing astrogliosis in a subject in need thereof, comprising administering glial fibrillary acidic protein (GFAP) or a fragment or breakdown product thereof, or an anti-GFAP antibody or GFAP-binding aptamer.

2. A method of treating a brain injury accompanied by astrogliosis in a subject in need thereof, comprising administering glial fibrillary acidic protein (GFAP) or a fragment or breakdown product thereof, or an anti-GFAP antibody or GFAP-binding aptamer.

3. The method of claim 1 or claim 2 wherein the subject suffers from traumatic brain injury, stroke, spinal cord injury, cerebral hemorrhage, chronic traumatic encephalopathy, Alzheimer's disease, Parkinson's disease, Huntington's disease, multiple sclerosis, amyotropic lateral sclerosis, frontotemporal dementia, tauopathy diseases, dementias, glioblastoma, vanishing white matter disease, epilepsy, hypoxic ischemic encephalopathy (HIE), neural damage due to drug or alcohol use or abuse, prion-related disease, peripheral neuropathy, diabetic neuropathy, and chemotherapy-induced neuropathy and neuropathic pain.

4. The method of claim 3, wherein the drug abuse is abuse of amphetamines or ecstasy (MDMA).

5. A pharmaceutical composition for immunization of a subject that has or is suspected of having astrogliosis, comprising:
(a) glial fibrillary acidic protein (GFAP) or a fragment thereof, an anti-GFAP antibody or a GFAP-binding aptamer; and
(b) a pharmaceutically acceptable carrier.

6. The pharmaceutical composition of claim 5, wherein the brain injury is caused by trauma.

7. The pharmaceutical composition of claim 5, wherein the brain injury is caused by a neurodegenerative disease.

8. The pharmaceutical composition of claim 5, which contains GFAP.

9. The pharmaceutical composition of claim 5, which contains an anti-GFAP antibody.

10. The pharmaceutical composition of claim 5, which contains a GFAP-binding aptamer.

11. A method of improving cognitive function in a subject in need thereof, comprising administering glial fibrillary acidic protein (GFAP) or a fragment or breakdown product thereof, or an anti-GFAP antibody or GFAP-binding aptamer.

12. The method of claim 11 or claim 12 wherein the subject suffers from traumatic brain injury, stroke, spinal cord injury, cerebral hemorrhage, chronic traumatic encephalopathy, Alzheimer's disease, Parkinson's disease, Huntington's disease, multiple sclerosis, amyotropic lateral sclerosis, frontotemporal dementia, tauopathy diseases, dementias, glioblastoma, vanishing white matter disease, epilepsy, hypoxic ischemic encephalopathy (HIE), neural damage due to drug or alcohol use or abuse, prion-related disease, peripheral neuropathy, diabetic neuropathy, and chemotherapy-induced neuropathy and neuropathic pain.

13. The method of claim 11 or claim 12, wherein administering comprises administering an anti-GFAP antibody.

14. A method of reducing GBDP in a subject in need thereof, comprising administering glial fibrillary acidic protein (GFAP) or a fragment or breakdown product thereof, or an anti-GFAP antibody or GFAP-binding aptamer, wherein the subject in need suffers from traumatic brain injury.

15. The method of claim 14, wherein administering comprises administering an anti-GFAP antibody.

16. A method of attenuating P-Tau/Total ratio in brain tissue associated with a traumatic brain injury in a subject, the method comprising administering glial fibrillary acidic protein (GFAP) or a fragment or breakdown product thereof, or an anti-GFAP antibody or GFAP-binding aptamer.

17. The method of claim 16, wherein administering comprises administering an anti-GFAP antibody.

18. A method of reducing circulatory Tau associated with a traumatic brain injury in a subject, the method comprising administering glial fibrillary acidic protein (GFAP) or a fragment or breakdown product thereof, or an anti-GFAP antibody or GFAP-binding aptamer.

19. The method of claim 18, wherein administering comprises administering an anti-GFAP antibody.
